(19) United States
(12) Patent Application Publication
(10) Pub. No.: US 2011/0129450 A1
(43) Pub. Date: Jun. 2, 2011

(54) METHOD OF PROMOTING NEUROGENESIS BY MODULATING SECRETASE ACTIVITIES

(76) Inventors: Orly Lazarov, Oak Park, IL (US); Archana Gadadhar, Chicago, IL (US); Michael P. Demars, Chicago, IL (US)

(21) Appl. No.: 13/057,097

(22) PCT Filed: Aug. 3, 2009
(86) PCT No.: PCT/US2009/052589

§ 371 Date: Feb. 1, 2011

Related U.S. Application Data
(60) Provisional application No. 61/085,519, filed on Aug. 1, 2008.
(60) Provisional application No. 61/085,513, filed on Aug. 1, 2008.
(60) Provisional application No. 61/093,109, filed on Aug. 29, 2008.

(51) Int. Cl. A61K 35/12; C12N 5/0797; A61K 31/713; A61K 31/7088; A61K 38/17; A61P 25/28; A61P 25/00; A61P 25/16

(52) U.S. Cl. 424/93.21; 435/377; 514/44 A; 435/375; 514/44 R; 514/17.7; 514/17.8

(57) ABSTRACT

This invention provides methods and reagents for promoting neurogenesis, by modulating neural stem cell proliferation and differentiation. Particularly, this invention provides methods and reagents for promoting neurogenesis in a patient's central nervous system where the patient suffers from an aging-related neurodegenerative disease. Specifically, the invention provides methods for promoting neurogenesis comprising modulating the α and/or γ-secretase activities.

This invention relates to and claims the benefit of priority to U.S. Provisional Application Ser. Nos. 61/085,513 filed on Aug. 1, 2008, 61/085,519 filed on Aug. 1, 2008, and 61/093,109 filed on Aug. 29, 2008. The disclosures of these three provisional applications are herein incorporated by reference in their entireties.

This invention is supported in part by Grant No. R01AG033570 from the National Institute On Aging (NIA). Thus, the United States Government has certain rights in the invention.

### BACKGROUND OF THE INVENTION

1. Field of the Invention

This application relates to the regulation of neural stem cell proliferation and differentiation. Specifically, the application relates to compositions, methods, and reagents useful for promoting neurogenesis and for treating a neurodegenerative disease, especially useful in patients with aging-related neurodegenerative diseases.

2. Description of Related Art

The hallmarks of Alzheimer's disease (AD) are amyloid deposits and neurofibrillary tangles. Amyloid deposition is the accumulation of amyloid beta protein in the brain. Amyloid beta (Aβ) is proteolytically processed from amyloid precursor protein (APP). APP is a transmembrane protein that is processed by a class of proteases called secretases into a variety of metabolites. The most widely studied APP metabolite is the amyloid beta (Aβ) peptide, which is produced by sequential processing of APP by β- and γ-secretases, and which has been implicated in the pathogenesis of AD. Cleavage of APP by α-secretase occurs at a site that resides between the β and γ cleavage sites and precludes Aβ formation. α-secretase cleavage leads to the production of the soluble APP fragment known as sαAPP and a membrane-tethered carboxyl-terminal fragment (CTF).

The enzymes associated with α-, β-, and γ-secretase activities are structurally distinct. Cleavage at the APP α-secretase site is accomplished by a variety of zinc metalloproteinases, which belong to the A Disintegrin And Metalloproteinase (ADAM) family; the enzymes ADAM9, ADAM 10, and ADAM 17 all demonstrate α-secretase activity (Postina, 2008, Curr. Alzheimer Res. 5: 179-86). In addition, a recently discovered aspartyl protease termed BACE2 exhibits α-secretase activity (Farzan et al., 2000, Proc. Natl. Acad. Sci. USA 97:9712-17). In contrast, a single aspartyl protease known as BACE1 (β-site APP cleaving enzyme 1) is associated with β-secretase activity (Cole et al., 2008, Curr. Alzheimer Res. 5: 100-20). Cleavage at the APP γ-site is performed by an aspartyl protease multiprotein complex, with the enzymes presenilin 1 (PS1) or presenilin 2 (PS2) comprising the catalytic core of the complex (Steiner, 2008, Curr. Alzheimer Res. 5: 147-57). The activities of the three secretases have been implicated in Alzheimer's disease pathology.

The majority of Alzheimer's disease patients is over 65 years of age, and is primarily characterized by progressive memory loss, cognitive decline and dementia. As of 2006, nearly 30 million people worldwide were estimated to suffer from symptoms of AD. Due to its high prevalence and incurability, AD is one of the most economically costly diseases to society. In unaffected individuals, APP is processed predominantly by α-secretase. However, decrease of α-secretase activity and/or enhanced O-secretase activity and/or dysfunction of γ-secretase may increase the production and/or fibrillogenic properties of the Aβpeptide (Cole et al., 2008; Steiner, 2008). The resulting accumulation of Aβ is linked to the debilitating and widespread neuronal death associated with AD (Crouch, 2008, Int. J. Biochem. Cell Biol. 40(2): 181-98).

In addition to AD, aging-related neurodegenerative diseases also include for example Parkinson's disease (PD), amyotrophic lateral sclerosis (ALS), Huntington's disease, and mild cognitive impairment (MCI), some of which are characterized by memory loss and dementia, and neuropathologically characterized by the appearance of amyloidosis.

The symptoms of PD, which often include tremors and loss of motor control and speech, are caused by a loss of dopamine-secreting nerve cells in the area of the brain known as the substantia nigra. ALS, known also as Lou Gehrig's Disease, is believed to be associated with degeneration of motor neurons, which causes muscle weakness, loss of voluntary muscle control, and muscle atrophy. HD is linked to a mutation in the huntingtin protein, which produces cellular changes in the brain and leads to mental decline and loss of coordination. MCI is also known as “incipient dementia,” and represents the transition between normal aging and Alzheimer's disease.

Currently, there is no effective treatment for these aging-related neurodegenerative diseases. AD treatments include cholinesterase inhibitors and glutamate N-methyl D-aspartate (NMDA) antagonists, none of which are able to prevent, halt, or reverse progression of neural loss as the underlying cause of the disease (Salloway and Correia, 2009, Cleve. Clin. J. Med 76(1): 49-58).

Until recently, neurogenesis was believed to occur only in the developing brain. However, neurons have since been shown to originate continuously throughout adulthood from neural stem cells, predominantly in two regions of the brain: the subventricular zone (SVZ) along the lateral ventricles, and the subgranular zone (SGZ) of the dentate gyms (DG), which resides in the hippocampus. However, enhancing endogenous neurogenesis as a therapeutic approach for neurodegenerative disease is yet to be shown.

In theory, aging-related neurodegenerative diseases might be treated by supplementing patients with neural stem cell (NSC) grafts. However, the implantation of allogeneic stem cells carries with it a variety of complications and obstacles, including the possibility of tissue rejection, ethical issues affiliated with using fetal tissue sources, and the high labor and financial costs associated with the need of individually tailored therapies for each patient.

Therefore, there is a need in the art for better therapeutic means to treat neurodegenerative diseases, especially aging-related neurodegenerative diseases in humans.

### SUMMARY OF THE INVENTION

This invention provides methods and reagents for promoting neurogenesis. Specifically, the application relates to compositions, methods, and reagents useful for promoting neural stem cell proliferation and differentiation, especially useful in patients with aging-related neurodegenerative diseases.

In one aspect, the invention provides methods of promoting neural stem cell proliferation comprising the step of increasing α-secretase activity in the neural stem cell. In certain embodiments, the α-secretase activity is increased by providing exogenous expression of a gene that possesses α-secretase activity in the neural stem cell. In certain other embodiments, the gene is ADAM9, ADAM10, BACE2 or ADAM17; and in certain particular embodiments, the gene is ADAM10. In yet other embodiments, the neural stem cell expresses amyloid precursor protein (APP).

In another aspect, the invention provides methods of promoting neural stem cell proliferation comprising the step of contacting the neural stem cell with a cell that expresses a protein having α-secretase activity. In certain embodiments the cell that contacts the neural stem cell expresses an exogenous gene that possesses α-secretase activity. In certain embodiments, the gene is ADAM9, ADAM10, BACE2 or ADAM17. In certain particular embodiments, the gene is ADAM10. In yet other embodiments, the cell that contacts the neural stem cell expresses APP.

In another aspect, the invention provides methods of promoting neural stem cell proliferation in a subject's central nervous system (CNS) comprising the step of increasing α-secretase activity in the subject's CNS. In certain embodiments, the α-secretase activity is increased in a neural stem cell in the subject's CNS. In certain other embodiments, the α-secretase activity is increased by providing exogenous expression of a gene that possesses α-secretase activity in the neural stem cell in the subject's CNS. In certain embodiments, the gene is ADAM9, ADAM10, BACE2 or ADAM17. In certain particular embodiments, the gene is ADAM10. In yet other embodiments, the neural stem cell expresses APP.

In yet another aspect, the invention provides methods of promoting neural stem cell proliferation in a subject's CNS by increasing α-secretase activity in the subject's CNS, comprising the step of contacting the subject's CNS with a cell that expresses a gene having α-secretase activity. In certain embodiments, the cell that contacts the subject's CNS is a cell derived from the subject. In certain other embodiments, the α-secretase activity is increased by providing exogenous expression of a gene that possesses α-secretase activity in the cell that contacts the subject's CNS. In certain embodiments, the gene is ADAM9, ADAM10, BACE2 or ADAM17. In certain particular embodiments, the gene is ADAM10. In yet other embodiments, the cell that contacts the subject's CNS expresses APP.

In a further aspect, the invention provides methods of promoting neurogenesis in a subject's CNS, comprising the step of increasing α-secretase activity in the subject's CNS. In certain embodiments, the α-secretase activity is increased by providing exogenous expression of a gene that possesses α-secretase activity in the subject's CNS. In certain other embodiments, the α-secretase activity is increased in a neural stem cell in the subject's CNS. In certain embodiments, the α-secretase activity is increased by providing exogenous expression of a gene that possesses α-secretase activity in a neural stem cell in the subject's CNS. In certain embodiments, the gene is ADAM9, ADAM10, BACE2 or ADAM17. In certain particular embodiments, the gene is ADAM10.

In another aspect, the invention provides methods of promoting neurogenesis in a subject's CNS comprising the step of contacting the subject's CNS with a cell that expresses a gene having α-secretase activity. In certain embodiments, the cell is a cell derived from the subject. In certain other embodiments, the cell expresses exogenously a gene that possesses α-secretase activity. In certain embodiments, the gene is ADAM9, ADAM10, BACE2 or ADAM17. In certain particular embodiments, the gene is ADAM10. In yet other embodiments, the cell that contacts the subject's CNS expresses APP.

In yet another aspect, the invention provides methods of treating a neurodegenerative disease in a subject comprising the step of increasing α-secretase activity in the subject's CNS wherein the increased α-secretase activity results in increased neural stem cell proliferation in the subject's CNS. In certain embodiments, the α-secretase activity is increased by providing exogenous expression of a gene that possesses α-secretase activity in the subject's CNS. In certain other embodiments, the α-secretase activity is increased by providing exogenous expression of a gene that possesses α-secretase activity in a neural stem cell in the subject's CNS. In certain embodiments, the gene is ADAM9, ADAM10, BACE2 or ADAM17. In certain particular embodiments, the gene is ADAM10.

In a further aspect, the invention provides methods of treating a neurodegenerative disease in a subject comprising the step of contacting the subject's CNS with a cell that expresses a protein having α-secretase activity. In certain embodiments, the cell that contacts the subject's CNS is a cell derived from the subject. In certain other embodiments, the cell expresses exogenously a gene that possesses α-secretase activity.

In certain advantageous embodiments, the neurodegenerative disease is an aging-related neurodegenerative disease. In certain particular embodiments. the aging-related neurodegenerative disease is Alzheimer's Disease, dementia, Parkinson's Disease, Huntington's disease, amyotrophic lateral sclerosis (ALS), or mild cognitive impairment (MCI).

In another aspect, the invention provides methods of inducing differentiation in a neural stem cell comprising the step of decreasing γ-secretase activity in the neural stem cell. In certain embodiments, the γ-secretase activity is decreased by a γ-secretase inhibitor. In certain particular embodiments, the γ-secretase inhibitor is a presenilin-1 (ps-1) siRNA.

In a further aspect, the invention provides methods of decreasing neural stem cell proliferation comprising the step of decreasing γ-secretase activity in the neural stem cell. In certain embodiments, the γ-secretase activity is decreased by a γ-secretase inhibitor. In certain particular embodiments, the γ-secretase inhibitor is a presenilin-1 (ps-1) siRNA. In certain other embodiments, the proliferation of neural stem cell is decreased in a subject's CNS.

In yet another aspect, the invention provides methods of inducing neural differentiation comprising the step of decreasing γ-secretase activity in a subject's CNS. In certain embodiments, the γ-secretase activity is decreased in a neural stem cell in the subject's CNS. In certain other embodiments, the γ-secretase activity is inhibited by a γ-secretase inhibitor. In certain particular embodiments, the γ-secretase inhibitor is a presenilin-1 (ps-1) siRNA.

In another aspect, the invention provides methods of promoting neurogenesis in a subject comprising a step of decreasing γ-secretase activity in the subject's CNS. In certain embodiments, the γ-secretase activity is decreased in a neural stem cell in the subject's CNS. In certain other embodiments, the γ-secretase activity is inhibited by a γ-secretase inhibitor. In certain particular embodiments, the γ-secretase inhibitor is a presenilin-1 (ps-1) siRNA.

In yet another aspect, the invention provides methods of treating neurodegenerative disease in a subject comprising a step of decreasing γ-secretase activity in the subject's CNS wherein the decreased γ-secretase activity results in increased neural differentiation in the subject's CNS. In certain embodiments, the γ-secretase activity is decreased in a neural stem cell in the subject's CNS. In certain other embodiments, the γ-secretase activity is inhibited by a γ-secretase inhibitor. In certain particular embodiments, the γ-secretase inhibitor is a presenilin-1 (ps-1) siRNA. In certain embodiments of the one or more aspects of the invention described herein, the γ-secretase inhibitor is a presenilin-2 (ps-2) inhibitor.

In yet another aspect, the invention provides methods of promoting neurogenesis in a subject comprising contacting the subject's CNS with the a form of the secreted amyloid precursor protein (sαAPP). In certain embodiments, the sαAPP promotes neural stem cell proliferation in the subject's CNS.

In a further aspect, the invention provides methods of treating a neurodegenerative disease in a subject, said method comprising a step of contacting the subject's CNS with sαAPP.

In certain advantageous embodiments of the above one or more aspects, the neurodegenerative disease is an aging-related neurodegenerative disease. In certain particular embodiments. the aging-related neurodegenerative disease is Alzheimer's Disease, dementia, Parkinson's Disease, Huntington's disease, amyotrophic lateral sclerosis (ALS), or mild cognitive impairment (MCI).

In certain embodiments of the above one or more aspects, the subject is a human.

Specific embodiments of the present invention will become evident from the following more detailed description of certain embodiments and the claims.

### BRIEF DESCRIPTION OF THE DRAWINGS

FIG. 1 shows bar graphs indicating reduced proliferation of neural stem cells in the sub-ventricular zone (SVZ) of knockout mice that are amyloid precursor protein heterozygous (APPHETKO) or homozygous (APPHOMKO) compared to wild-type (APPWT) mice. Stereological analysis of (A) the number of proliferating neural stem cells (labeled with bromodeoxyuridine, BrdU+), (B) the number of newly differentiating neurons using doublecortin (DCX) as an early neuronal marker (BrdU+DCX+), and (C) the number of newly differentiating astrocytes (using glial fibrillary acidic protein (GFAP) as an astrocyte marker (BrdU+GFAP+); *, p<0.05 using a standard Student's t-test. (D) Clonogenic assay of neurospheres derived from APPKO (HOMOKO) and APPWT brains; **, p<0.0001 using a standard Student's t-test.

FIG. 2 depicts photographs of immunoblot analysis showing steady state levels of secreted APP (sAPP) in the SVZ of adult mice. (A) Immunoblot blot analysis of expression levels of sAPP and full-length APP (FL-APP) in the SVZ of APP homozygous knockout (APPHOMKO), APP heterozygous knockout (APPHETKO) mice and APP wild type (APPWT) mice using an antibody (the 22C11 antibody) specific for the N-terminus of APP. (B) Immunoblot analysis of brain extracts depleted with FL-APP using an antibody raised against the C-terminus of APP (the 369 antibody), followed by detection of sAPP using the 22C11 antibody.

FIG. 3 shows (A) a bar graph representing results of proliferation assays of NSC treated with the ADAM inhibitor GM6001 or its inactive analog GM6001NK (10 μM or 1 mM); **, p<0.01. APPWT CM represents proliferation results of GM6001-treated cells supplemented with APPWT-conditioned media. (B) Immunoblot analysis of sAPP levels in conditioned media from GM6001- and GM6001NK-treated NSC using the 22C11 antibody.

FIG. 4 depicts a bar graph showing the results of proliferation assays of adult NSCs that were singly dissociated, plated for 8 days, then treated with GM6001, the inactive analog GM6001NK, treated with GM6001 and supplemented with conditioned medium from neuroblastoma N2a cells expressing wild type APP (sαAPP-enriched conditioned media) or supplemented with conditioned medium from 192Swe N2a cells (sβAPP-enriched conditioned media).

FIG. 5A shows a schematic diagram of a recombinant lentiviral vector expressing shRNA under the control of the U6 pol III for silencing PS1, having green fluorescent protein (GFP) under the control of a CMV promoter for the identification of transduced cells. FIG. 5B shows an image of Nissl stained sagittal mouse brain section. Arrows indicate sites of lentiviral sterotaxic injection: SGL=subgranular layer and SVZ=subventricular zone. (LV=lateral ventricle, RMS=rostral migratory stream, OB=olfactory bulb)

FIGS. 6A and 6B show photographs of immunoblot analysis of PS1 expression in vitro and in vivo following transduction of NSCs with lentiviral vectors expressing PS1 siRNA. FIG. 6C shows microphotographs of confocal immunostaining of brain sections of adult C57/B16 mice six weeks following stereotaxic injection of lentiviral vectors expressing GFP and PS1 siRNA into the SG (SGL, panels a-d) or into the SVZ (panel e). GFP-positive staining (panel a) and the mature neuron marker NeuN-positive staining (panel b) was merged and shown in panel c. Panel d shows the image of the whole hippocampus stained with antibodies to GFP and NeuN. Scale bar=150 μm (panels a-c), 250 μm (panel d) and 75 μm (panel e).

FIGS. 7A-7D depict bar graphs showing the results of a stereological analysis of the number of lentiviral vector-transduced cells that were undergoing proliferation (GFP+BrdU+) (FIG. 7A), transduced newly-differentiating neurons using a late neuronal marker β-tubulin (GFP+BrdU+β-tubulin+) (FIG. 7B), transduced newly-differentiating astrocytes (GFP+BrdU+GFAP+) (FIG. 7C) and transduced newly-differentiating neurons using an early neuronal marker DCX (GFP+BrdU+DCX+) (FIG. 7D).

FIG. 7E, panels a and b show representative photomicrographs of confocal immunofluorescence staining for BrdU, DCX and GFAP in the SVZ (panel a) or SGL (panel b) region of brain sections from adult mice. BrdU: single arrows; DCX: double arrows; and GFAP: dotted arrows. Panels c and d show GFP+BrdU+immunostaining (panel c) or GFP+NeuN+ immunostaining (panel d) in the SGL of mice three weeks following stereotaxic injection of lentiviral PS1 siRNA vectors. NeuN+: dotted arrow; GFP+: single arrow. Panels e and f show images of GFP+BrdU+ immunostaining (panel e) or GFP+NeuN+ immunostaining (panel f) in the granule layer (GL) of the dentate gyms (DG) in mice six weeks after lentiviral transduction. GFP/NeuN double-stained cells are marked by the double arrow, and representative NeuN single-positive cells are marked by single arrows (panel f). Panel g shows GFP+ immunostaining in the granule layer of the DG in mice six weeks after transduction of the lentiviral vectors. Dotted arrows indicate NeuN staining; and single arrows indicate GFP staining Panel h shows image of GFP/BrdU/DCX triple-immunostaining in the SGL of mice six weeks after transduced with the lentiviral vectors (double arrow: GFP+BrdU+DCX+ cell; dotted arrow: a region that is positive for DCX only). Panel i shows GFP/GFAP double immunostaining in the SGL of mice six weeks after being transduced with lentiviral vectors (double arrow: GFP+GFAP+ cell). Scale bar=75 μm (panels a,b), 50 μm (panels c.d), 65 μm (panel e), 45 μm (panels f,h,i) and 85 μm (panel g). Panels c-i show images of immunostained brain sections from mice transduced with PS1 siRNA vector.

FIG. 8 shows the effects of the γ-secretase inhibitor L-685,458 on neural stem cell proliferation and differentiation. FIG. 8A is a bar graph showing the results of proliferation assays of neurosphere cultures transduced with lentiviral vectors expressing an irrelevant siRNA (IR siRNA), siRNA for PS1 targeting, or neurospheres treated with the γ-secretase inhibitor L-685,458. The rate of proliferation is presented as percentage of proliferation of DMSO-treated cells (*, p<0.05; **, p<0.01 by standard Student t-test). FIG. 8B shows phase contrast images of differentiation assays showing neural stem cells differentiating following treatment with L-685,458 (lower panel), or the vehicle DMSO (upper panel). FIG. 8C shows a bar graph representing the number of differentiated cells after a two-day DMSO- or L-685,458 treatment (*, p<0.05, standard Student's t-test). FIG. 8D depicts a bar graph indicating the number of neurospheres formed from singly dissociated neurosphere cultured cells following a two-day treatment with L-685,458 (*, p<0.05, standard Student's t-test). FIG. 8E shows photomicrographs of confocal immunofluorescence staining of cells treated with vehicle or L-685,458 with antibodies against nestin and GFAP. Scale bar=75 μm. FIG. 8F shows photomicrographs of confocal immunofluorescence staining of control or L-685,458-treated neurospheres after being cultured on laminin using antibodies specific for β-tubulin (single arrows), GFAP (dotted arrows) and counterstained with DAPI. FIG. 8G depicts a bar graph showing the length of processes from the middle of the soma to the axon tip in GFAP+ cells of control or L-685,458-treated cells. FIG. 8H depicts a bar graph showing the results of stereological analysis indicating a decrease in the number of cells that were GFAP+Nestin+DCX+ after L-685,458 treatment.

FIG. 9A depicts photomicrographs of confocal microscopy images of SVZ-derived neurosphere cells three days after transduction with lentiviral vectors expressing PS1 siRNA and GFP: GFP positive neurospheres (panels a, d), nestin-positive neurospheres (panels b, e), and the merged images showing GFP+nestin+ neurospheres (panels c, f). FIG. 9B depicts photographs of immunoblot analysis of PS1 and GFAP expression in protein extract of neurosphere cultures transduced with lentiviral preparations expressing IR siRNA or PS1 siRNA.

### DETAILED DESCRIPTION OF THE INVENTION

All publications, patents and published patent applications cited herein are hereby expressly incorporated by reference for all purposes. Within this application, unless otherwise stated, the techniques utilized may be found in any of several well-known references such as: Molecular Cloning: A Laboratory Manual (Sambrook et al., 1989, Cold Spring Harbor Laboratory Press).

As used herein, the singular forms “a”, “an” and “the” include plural referents unless the context clearly dictates otherwise. For example, reference to a “lentiviral vector” means one or more lentiviral vectors.

As used herein, the terms “polynucleotide”, “nucleotide”, “oligonucleotide”, and “nucleic acid” may be used interchangeably to refer to single stranded nucleic acid comprising DNA, RNA, derivative thereof, or combination thereof.

As used herein the term “neural stem cells,” “neural progenitor cells” or “NSCs” refers to self-renewing, multipotent cells that can differentiate into neurons, astrocytes, oligodendrocytes, glial cells, and other neural-lineage cells, which in turn give rise to the nervous system.

This invention provides methods and reagents for promoting or modulating neurogenesis, specifically neural stem cell proliferation or differentiation. In certain embodiments, the invention provides methods for modulating neural stem cell proliferation or differentiation in a subject's central nervous system (CNS). In certain particular embodiments, the invention provides methods and reagents for modulating neural stem cell proliferation and differentiation by increasing α-secretase activities and/or decreasing γ-secretase activity. The invention also provides methods and reagents for increasing neural stem cell proliferation and differentiation in a human suffering from a neurodegenerative disease.

While the synaptic connections involved in neural circuits are continuously altered throughout the life of the individual (due to synaptic plasticity and cell death), neurogenesis, the generation of new neurons, had been thought to be complete early in the postnatal period. The discovery of NSCs in the adult brain (see, Gould et al., 1999, Science 286: 548-552) brought new understanding of neurogenesis, as the presence of NSCs in the adult brain suggests that regeneration of neurons can occur throughout life. Nevertheless, age, physical and biological trauma or neurodegenerative disease-associated loss of brain function can far outweigh any potential restorative capacity of endogenous neurogenesis.

It has been reported that adult neurogenesis occurs predominantly in the subventricular zone (SVZ) along the lateral ventricles, and the subgranular zone (SGZ) of the dentate gyms (DG), which resides in the hippocampus. A component of the brain's limbic system, the hippocampus plays an integral role in long term memory, spatial memory, and the formation of new memories. The hippocampus is one of the first regions to suffer damage during the progression of AD, which accounts for the disorientation and memory loss symptomatic of the early stages of the disease. In normal mice, neurons generated in the SVZ travel to the olfactory bulb, which is a region in the brain that often shows signs of damage during the progression of AD.

The hippocampus is also one of several regions, including the striatum, the substantia nigra, and the cerebral cortex, that are damaged by Huntington's disease, which is often manifested by the decline of mental abilities into dementia.

There has been evidence indicating that hippocampal adult neurogenesis is important for learning and memory. See Kempermann et al. 2004, Curr. Opin. Neurogiol. 14:186-91. It has also been hypothesized that neurogenesis in the adult brain originates from neural stem cells. Thus, increased neural stem cells proliferation would permit increased neural differentiation to generate neurons and astrocytes to replenish the lost neural cells commonly seen in the progression of neurodegenerative diseases.

The levels of α-secretase decline during the aging process. Both α- and γ-secretases are involved in the regulation of amyloid beta production, which is known to be associated with the progression of Alzheimer's disease. However, the roles of α- and γ secretases in neurogenesis were not known and the link between amyloid beta and memory loss remains uncertain. It is believed that neurogenesis in adulthood produces newly differentiated neural and glial cells from a pool of neural stem cells. It was unexpectedly discovered in the instant invention that α-secretase and γ-secretase affected the neurogenesis process, specifically, by affecting neural stem cell proliferation and neural differentiation.

Thus, in one aspect, this invention provides methods of promoting or increasing neural stem cell proliferation comprising the step of increasing α-secretase activity in the neural stem cell. In certain embodiments, α-secretase activity is increased by providing the neural stem cell with exogenous polynucleotide molecules that encode a protein having α-secretase activity.

As used herein, the term “α-secretase activity” refers to an enzymatic activity, or multiple enzymatic activities, that effectuate the proteolytic cleavage of APP at the α-secretase cleavage site. Several metalloproteases, such as ADAM9, ADAM10, and ADAM17, and a newly discovered aspartyl protease BACE2, possess the α-secretase activity. The phrase “a cell expressing α-secretase activity” refers to a cell that comprises one or more genes that express one or more proteins possessing α-secretase activity.

In certain embodiments of this aspect, neural stem cell proliferation is promoted by increasing α-secretase activity in the neural stem cell itself. In certain embodiments, the α-secretase activity is increased by introducing an exogenous gene having α-secretase activity into the neural stem cell.

In certain embodiments, neural stem cell proliferation is promoted by increasing α-secretase activity in the neural stem cell in vitro. Said in vitro methods of promoting neural stem cell proliferation facilitate improved neuroreplacement therapies: such in vitro proliferating neural stem cells can be transplanted to a subject in need thereof. In certain embodiments, the neural stem cell is isolated from the subject, and thus autologous to the subject.

In certain advantageous embodiments, the neural stem cell is derived from bone-marrow mesenchymal stem cells or other adult stem cells isolated from the subject in need of neuroreplacement therapy. Pluripotent mesenchymal stem cells can be induced or modified to become neural stem cells, committed to neural-lineage differentiation, as shown inter alia in U.S. Patent Application Publication Nos. 2009/0219898, 2003/0148513, and 2003/0139410. Thus, in accordance with this aspect of the invention, mesenchymal stem cells can be isolated from a subject, and induced to become neural stem cells in vitro by methods known in the art. Neural stem cell proliferation is induced by increasing the α-secretase activity in the neural stem cell as described herein. Alternatively, neural stem cell proliferation is enhanced by contacting the neural stem cell with a cell that expresses a gene that possesses α-secretase activity. In certain particular embodiments, the neural stem cell or the cell contacting the neural stem cell expresses APP.

In certain embodiments, α-secretase activity is increased by providing exogenous expression of a gene that possesses α-secretase activity in the subject's CNS. Genes that possess α-secretase activity suitable for use in this aspect of the invention include without limitation ADAM9 (for example, GenBank Accession Nos. for human ADAM9: BC143923, SEQ ID NOs:1 and 2; for mouse ADAM9: BC047156, SEQ ID NOs:3 and 4), ADAM10 (for example, GenBank Accession Nos. for human ADAM10: BC126253, SEQ ID NOs:5 and 6; for mouse ADAM10: BC168390, SEQ IDs NO:7 and 8), ADAM17 (for example, GenBank Accession Nos. for human ADAM17: BC136783, SEQ ID NOs:9 and 10; for mouse ADAM17: BC145270, SEQ ID NOs:11 and 12), and BACE2 (for example, GenBank Accession Nos. for human BACE2: BC014453, SEQ ID NOs:19 and 20; for mouse BACE2: BC131947, SEQ ID NOs:21 and 22).

Methods of measuring neural stem cell proliferation are routine practice to one of ordinary skill in the art and are further described in the instant application. Commonly employed methods analyzing neural stem cell proliferation include without limitation neurosphere self-renewal and proliferation (clonogenic) assays and BrdU-pulse labeling and colorimetric assays as described herein.

In certain other embodiments of this aspect, the invention provides methods of promoting or increasing neural stem cell proliferation comprising contacting a neural stem cell with a cell that expresses α-secretase activity. In certain other particular embodiments, α-secretase activity is increased by providing exogenous expression of a gene that possesses α-secretase activity in the cell contacting the neural stem cell. In certain particular embodiments, α-secretase activity is increased in a subject's CNS. In certain other particular embodiments, α-secretase activity is increased by providing exogenous expression of a gene that possesses α-secretase activity in the subject's CNS.

It is within the knowledge of one of ordinary skill in the art to select methods and reagents for detecting and analyzing α-secretase activity in a cell. Methods suitable for use in verifying α-secretase activity in a cell include without limitation direct assays for the production of sαAPP, and indirect assays for the expression of one or more genes that account for the α-secretase activity. The latter can be achieved by Northern bolt analysis or RT-PCR using primer or probe sequences derived from the sequences of one or more genes that possess α-secretase activities as described herein; and immunoblot analysis, using antibodies specific for one or more genes that are known to possess α-secretase activity, such as ADAM9, ADAM10, BACE2 and ADAM17. Commercially available antibodies can be obtained from, for example, Santa Cruz Biotechnology (monoclonal antibody specific for ADAM9, catalog No. M901L; polyclonal antibody to ADAM17, catalog No. TACE C-15, Santa Cruz, Calif.) and Abcam (polyclonal antibody to ADAM10, catalog No. ab1997, Cambridge, Mass.).

In certain particular embodiments, the α-secretase activity is measured directly by measuring the production of sαAPP or using a fluorogenic substrate-based assay (α-secretase substrate II, catalog No. 565767, Merck, Whitehouse Station, N.J.), as indicated by the manufacturer.

It is known that at least two strategies can be used to achieve transgene expression in a target cell population, i.e., in vivo or ex vivo gene therapy, the principles and procedures for both of which are well-known in the art (Blesch et al., 2004, Yonsei Medical Journal 45: 28-31; Snyder et al. 1997, Neurobiology Disease 4:69-102). For ex vivo gene therapy, preferably autologous cells are removed from the subject, genetically modified to express an exogenous gene in vitro, and the recombinant cells are implanted into the subject at the desirable location. In vivo gene therapy involves the direct injection of viral or non-viral vectors into the target tissue, and thus bypasses the need for an autologous cell graft (see U.S. Pat. No. 7,244,423).

In certain aspect, the invention provides methods of promoting neural stem cell proliferation in a subject's CNS comprising the step of increasing α-secretase activity. Suitable gene therapy vectors for use in the invention comprise any agent that comprises a polynucleotide, and the vector can deliver the gene of interest to a target tissue, thereby leading to the expression of the gene of interest. In certain embodiments of this aspect, the gene of interest is a gene that possesses α-secretase activity, including without limitation ADAM9, ADAM10, BACE2 and ADAM17.

The polynucleotide can be any genetic construct made from nucleic acids, including DNA or RNA. Suitable genetic constructs directing the expression of gene of interest include without limitation plasmid DNAs and engineered attenuated or inactivated retroviruses. The expression of the gene of interest can be positioned under the control of a promoter either constitutively active or inducible in most mammalian cells, or constitutively active or inducible in particular tissues. In certain embodiments of this invention, the promoter for driving α-secretase expression is a neural-specific promoter such as nestin promoter (Dahlstrand et al., 1992, J. Cell Sci. 103:589-597, GenBank Accession No. NM_{—}006617) or SRY (sex determining region Y)-box 2 (Sox-2) promoter (Stevanovic et al., 1994, Mammalian Genome 5:640-642, GenBank Accession No. NM_{—}003106). Cell-specific gene expression can be achieved by expression the gene under cell-type specific promoter.

The vector can be a virus, such as papovirus, lentivirus, adenovirus, vaccinia virus, adeno-associated virus, herpesvirus, and retrovirus, and is preferably lentivirus. Non-viral gene therapy methods may also be used with the methods of the invention. Naked plasmids, lipid-nucleic acid complexes (known as lipoplexes), and complexes of synthetic polymers or amino acids with nucleic acids are all methods which have been used to successfully target cells with exogenous genes of interest.

Because most adult mammalian brain cells do not divide, in certain particular embodiments, a viral vector to be used in the methods of the invention can transfect and facilitate expression of the transgene in non-dividing cells. Such expression vectors include adeno-associated virus, lentivirus, herpesvirus, alpha viruses, and pox virus. The viral vectors can be injected to the desirable locations intracerebrally. Viral vectors that allow expression of a transgene without the pathogenesis associated with the viral proteins from the viral vectors are known in the art (see e.g., Naldini et al. 1996, Science 272: 263-7).

As used herein the terms “viral vector” and “virus” are used interchangeably when referring to the gene transfer vehicle that delivers into a cell the desirable gene such as a gene that possesses α-secretase activity. In this context, the term “viral vector” refers to a packaged recombinant virus that is used as a delivery vehicle for gene transfer.

In certain other embodiments, the invention provides methods of promoting neural stem cell proliferation in a subject's CNS comprising the step of contacting the subject's CNS with a cell that expresses α-secretase activity. In certain embodiments, the cell expresses exogenous α-secretase activity. The methods for generating a recombinant cell expressing an exogenously-introduced gene of interest, such as a gene that possesses α-secretase activity, is known in the art. In certain embodiments, transfer of an expression vector into a selected host cell can be accomplished by well-known methods including transfection, specifically calcium chloride-mediated transfection, viral infection, electroporation, microinjection, lipofection, DEAE dextran-mediated transfection, or other known techniques. In certain embodiments, the exogenous expression is achieved by transient transfection; while in other particular embodiments, the exogenous expression is achieved by stable transfection. For the purpose of stable transfection, the DNA construct preferably contains a selectable marker, such as neo or hyg B, which confers resistance to a selection agent, such as geneticin (an analog of neomycin) or hygromycin, respectively. Furthermore, the transfection protocol influences the stability of transfection. For example, one skilled in the art can use calcium phosphate, electroporation, and viral infection to yield stably transfected recombinant cells, whereas lipofection is typically associated with transient transfection.

The selection of a particular vector will depend on the gene therapy strategy (i.e. in vivo or ex vivo) and, in the case of ex vivo gene therapy, the type of host cells used, because certain vectors are more effective in certain cell types than in others. A number of suitable host cells for use in intracerebral ex vivo gene therapy are known in the art, including fibroblasts, neurons, glial cells, particularly astrocytes, oligodendrocytes, glial progenitors, neural stem cells, bone marrow-derived hematopoietic stem cells, myoblasts, and activated macrophages. See Snyder et al., 1997, Neurobiology of Disease 4: 69-102). Selection of a particular cell type by one skilled in the art would be based on several factors, including (1) the extent of damage to the part of the body from which the cells were removed, (2) the ability of the cells to survive in the new location, (3) the likelihood that the cells could be successfully genetically manipulated in vitro to produce and secrete the protein coded by the exogenous gene of interest in sufficient quantities, and (4) the ability of the cells in the autologous graft, once re-implanted, to remain inert and innocuous in their new location.

In a further aspect, the invention provides methods of promoting neural stem cell proliferation in a subject comprising a step of contacting the subject's CNS with a cell that expresses a gene that possesses α-secretase activity and expresses the amyloid precursor protein (APP). In yet another aspect, the invention provides a method of promoting neural stem cell proliferation in a subject comprising a step of contacting the subject's CNS with a cell that expresses a protein having α-secretase activity and expresses the amyloid precursor protein (APP).

In certain embodiments, the expression of APP in the cell can be verified by methods well known in the art including without limitation Northern blot analysis and RT-PCT using probe and primer sequences derived from the APP sequence that is known in the art (GenBank Accession Nos. for human APP: BC065529; for mouse APP: BC070409). In certain other embodiments, the expression of APP can be detected by using APP specific antibody such as the commercially available polyclonal antibody obtained from Abcam (catalog #Ab15272, Cambridge, Mass.). In certain embodiments, the cell expresses exogenous α-secretase. In certain other embodiments, the cell expresses exogenous APP.

In yet another aspect, the invention provides a method of promoting neurogenesis in a subject's CNS, said method comprising a step of increasing α-secretase activity in the subject's CNS. In a further aspect, the invention provides a method of promoting neurogenesis in a subject's CNS comprising a step of contacting the subject's CNS with a cell that expresses α-secretase activity and APP.

As used herein, a “subject” refers to an animal with a central nervous system, especially a mammal, most particularly a human. In certain particular embodiments, the subject is a human suffering from an aging-related neurodegenerative disease.

As used herein, the phrase “a cell derived from the subject” refers to a cell removed and/or isolated from the subject, and thus the cell is autologous to the subject. Suitable cells that can be removed, isolated and used in the instant invention include without limitation fibroblasts, Schwann cells, endothelial cells, neurons, glial cells, astrocytes, oligodendrocytes, glial progenitors, neural stem cells, bone marrow-derived hematopoietic stem cells, myoblasts, and activated macrophages. See e.g., Gage et al., 1987, Neuroscience 23:795-807; Senut et al. 1996, In: Genetic Manipulation of the Nervous System, pp. 181-202, Academic Press, San Diego, Calif. In certain embodiments, the cell is syngeneic or isogeneic primary cells with respect to the subject, such as primary cells removed and isolated from the subject's identical twin. In certain other embodiments, the cell is heterologous to the subject but non-immunogenic, or with reduced immunogenicity.

In certain alternative embodiments, α-secretase activity is increased by contacting the cell with an activator of α-secretase activity. Activators that promote α-secretase activity are known in the art include without limitation EGF; FGF; NGF; VGEF; chemokines such as chemokine (C-X3-C motif) ligand 1 (CX3CL1, also known as fractalkine in humans and neurotactin in mice, GenBank Accession Nos. NP_{—}002987 and NP_{—}033168, respectively); protein kinase C activators, such as bryostatin, benzolactam, and LQ12; all-trans-retinoic acid; calcium ionophore A23187; activator of the tyrosine kinase pathway; acetylcholinesterase inhibitor donepezil; MAP kinase pathway activators; protein kinase A; regulators of clathrin-mediated endocytosis such as endophilin; N-methyl D-Aspartate (NMDA) receptor activators; monoamine oxidase inhibitor such as deprenyl; and muscarinic receptor agonists such as talsaclidine and carbachol. See, for example, Lichtenthaler, 2006, Neurodegener Dis. 3:262-269; Bandyopadhyay et al., 2007, Curr. Med. Chem. 14:2848-2864; Hock et al., 2003, Amyloid. 10:1-6; Yang et al., 2009, Eur. J. Pharmacol. 610:37-41; Kozikowski et al., 2009, ChemMedChem 4:1095-1105; Hoey et al., 2009, J. Neurosci 29:4442-4460; Koryakina et al., 2009, FEBS J. 276:2645-2655; and Wolf et al., 1995, J. Biol. Chem. 270:4916-4922. In certain particular embodiments, α-secretase activity is increased in a subject's central nervous system (CNS).

In addition to the effects of α-secretase activity on neural stem cell proliferation, reagents and methods for promoting neural stem cell differentiation comprising reducing γ-secretase activity are disclosed herein.

Thus, in a further aspect, the invention provides methods of inducing differentiation in a neural stem cell comprising the step of decreasing γ-secretase activity. In another aspect, the invention provides methods of reducing neural stem cell proliferation comprising the step of decreasing γ-secretase activity. In yet another aspect, the invention provides methods of inducing differentiation in a neural stem cell or reducing neural stem cell proliferation in a subject's CNS. In a further aspect, the invention provides methods of promoting neurogenesis in a subject comprising a step of decreasing γ-secretase activity in the subject's CNS. In yet another aspect, the invention provides methods of treating neurodegenerative disease in a subject comprising the step of decreasing γ-secretase activity in the subject's CNS wherein the decreased γ-secretase activity results in increased neural differentiation in the subject's CNS.

As used herein the term “γ-secretase activity” refers to the enzymatic activity that is responsible for the proteolytic cleavage of the APP at the γ-secretase cleavage site. The γ-site cleavage is performed by an aspartyl protease multiprotein complex, with the enzymes presenilin 1 (PS1) or presenilin 2 (PS2) comprising the catalytic core of the complex (Steiner, 2008, Curr. Alzheimer Res. 5: 147-57). Inhibition of at least the core catalytic components PS1 and/or PS2 can result in the decreased γ-secretase activity.

In certain embodiments, the γ-secretase activity is inhibited by a γ-secretase inhibitor. In certain advantageous embodiments, the γ-secretase inhibitor is a presenilin-1 (PS-1) (for example, GenBank Accession Nos. for human PS-1: BC011729, SEQ ID NOs:13 and 14; for mouse PS-1: BC071233, SEQ ID NOs:15 and 16) siRNA. In certain other embodiments, the γ-secretase inhibitor is PS-2 siRNA (for example, catalog No. sc-155863, Santa Cruz Technologies, Santa Cruz, Calif.). In further embodiments of this aspect, the neurodegenerative disease is an aging-related neurodegenerative disease. In certain particular embodiments, the aging-related neurodegenerative disease includes without limitation Alzheimer's disease, dementia, Parkinson's disease, Huntington's disease, amyotrophic lateral sclerosis (ALS), or mild cognitive impairment (MCI).

In certain embodiments, the PS-1 siRNA can be expressed from a genetic construct; in certain advantageous embodiments, the PS-1 siRNA can be expressed from a lentiviral vector. Both the genetic constructs and the lentiviral vector can be injected intracerebrally into a subject's CNS as described herein.

Other suitable γ-secretase inhibitors for use in the instant invention include without limitation L-685,458, 31C-III, N—[N-3,5-difluorophenacetyl)-L-alanyl]-5-phenylclycine t-butyl ester (DAPT), LY450139 (Semagacestat) (Imbimbo et al., 2009, Curr. Opin. Investig. Drugs 10:721-30), BMS-299897, GSI-953, and ELN318463 (Tomita, 2009, Expert Rev. Neurother. 9: 661-79). In certain particular embodiments, the γ-secretase inhibitor is Semagacestat. Most suitable γ-secretase inhibitors for use in the instant invention preferably do not affect or inhibit Notch activity, the inhibition of which may lead to undesirable side effects upon long-term use of the inhibitors. It is further understood by one of skill in the art that therapeutic index for a γ-secretase inhibitor has to be calculated to determine the toxicity and suitable doses for administering the inhibitor into a subject in need thereof.

In yet another aspect, the invention provides methods for promoting neurogenesis in a subject comprising contacting the subject's CNS with the a form of the secreted amyloid precursor protein (sαAPP). In a further aspect, the invention provides methods for treating a neurodegenerative disease in a subject, said method comprising the step of contacting the subject's CNS with sαAPP. In certain embodiments of the aspect, the neurodegenerative disease is an aging-related neurodegenerative disease including without limitation Alzheimer's disease, dementia, Parkinson's disease, Huntington's disease, amyotrophic lateral sclerosis (ALS), and mild cognitive impairment (MCI). In certain embodiments of all of the above aspects of the invention, the subject is a human.

In certain embodiments, the therapeutic compound, such as an α-secretase activator, a γ-secretase inhibitor, or sαAPP, is administered to a subject through systemic injection. The blood-brain barrier functions to hinder effective delivery of certain therapeutic compounds to the brain and presents a challenge to treatment of brain disorders. The barrier restricts diffusion of microscopic objects and large or hydrophilic molecules; the barrier, however, allows diffusion of small hydrophobic molecules. Drug delivery across the blood-brain barrier can be achieved by temporarily disrupting the barrier by osmotic means or ultrasound-aided drug delivery, by utilizing endogenous carrier-mediated transporters or receptor-mediated transcytosis, or encapsulating drugs in liposomes. Patients with neurodegenerative diseases such as AD often have compromised or disrupted blood-brain barrier that permits easier passage of therapeutic compounds. In alternative embodiments, the blood-brain barrier is overcome by intracerebral injection or implantation of the therapeutic compound.

In certain embodiments, the sαAPP is injected in the denate gyms and/or SVZ of the brain. In certain particular embodiments, the sαAPP is tagged with a cell type-specific molecule, such as a cell type-specific antibody, for cell type-specific targeted delivery of sαAPP. In certain other embodiments, the sαAPP is injected systematically by intraperitoneal or intravenous injection. The systemically injected sαAPP can cross the blood-brain barrier in AD patients where the blood-brain barrier is compromised or disrupted.

In certain embodiments, the therapeutic compound is injected into a subject in conjunction with a pharmaceutical acceptable carrier, diluent or excipient known to one of skill in the art for modifying, maintaining, or preserving, in a manner that does not hinder the activities of the therapeutic compounds or molecules described herein, for example, pH, osmolarity, viscosity, clarity, color, isotonicity, odor, sterility, stability, rate of dissolution or release, adsorption, or penetration of the composition. Suitable formulation materials include, but are not limited to, amino acids (such as glycine, glutamine, asparagine, arginine, or lysine), antimicrobial compounds, antioxidants (such as ascorbic acid, sodium sulfite, or sodium hydrogen-sulfite), buffers (such as borate, bicarbonate, Tris-HCl, citrates, phosphates, or other organic acids), bulking agents (such as mannitol or glycine), chelating agents (such as ethylenediamine tetraacetic acid (EDTA)), complexing agents (such as caffeine, polyvinylpyrrolidone, betacyclodextrin, or hydroxypropyl-beta-cyclodextrin), fillers, monosaccharides, disaccharides, and other carbohydrates (such as glucose, mannose, or dextrins), proteins (such as serum albumin, gelatin, or immunoglobulins), coloring, flavoring and diluting agents, emulsifying agents, hydrophilic polymers (such as polyvinylpyrrolidone), low molecular weight polypeptides, salt-forming counterions (such as sodium), preservatives (such as benzalkonium chloride, benzoic acid, salicylic acid, thimerosal, phenethyl alcohol, methylparaben, propylparaben, chlorhexidine, sorbic acid, or hydrogen peroxide), solvents (such as glycerin, propylene glycol, or polyethylene glycol), sugar alcohols (such as mannitol or sorbitol), suspending agents, surfactants or wetting agents (such as pluronics; PEG; sorbitan esters; polysorbates such as polysorbate 20 or polysorbate 80; Triton; trimethamine; lecithin; cholesterol or tyloxapal), stability enhancing agents (such as sucrose or sorbitol), tonicity enhancing agents (such as alkali metal halides—preferably sodium or potassium chloride—or mannitol sorbitol), delivery vehicles, diluents, excipients and/or pharmaceutical adjuvants. See REMINGTON′S PHARMACEUTICAL SCIENCES (18th Ed., A. R. Gennaro, ed., Mack Publishing Company 1990).

The primary vehicle or carrier in a pharmaceutical composition may be either aqueous or non-aqueous in nature. For example, a suitable vehicle or carrier for injection may be physiological saline solution, or artificial cerebrospinal fluid. Optimal pharmaceutical compositions can be determined by a skilled artisan depending upon, for example, the intended route of administration, delivery format, desired dosage and recipient tissue. See, e.g., REMINGTON′S PHARMACEUTICAL SCIENCES, supra. Such compositions may influence the physical state, stability, and effectiveness of the composition.

The pharmaceutical composition to be used for in vivo administration typically is sterile and pyrogen-free. In certain embodiments, this may be accomplished by filtration through sterile filtration membranes. In certain embodiments, where the composition is lyophilized, sterilization using this method may be conducted either prior to or following lyophilization and reconstitution. In certain embodiments, the composition for parenteral administration may be stored in lyophilized form or in a solution. In certain embodiments, parenteral compositions generally are placed into a container having a sterile access port, for example, an intravenous solution bag or vial having a stopper pierceable by a hypodermic injection needle.

Once the pharmaceutical composition of the invention has been formulated, it may be stored in sterile vials as a solution, suspension, gel, emulsion, solid, or as a dehydrated or lyophilized powder. Such formulations may be stored either in a ready-to-use form or in a form (e.g., lyophilized) that is reconstituted prior to administration.

The effective amount of a pharmaceutical composition of the invention to be employed therapeutically will depend, for example, upon the therapeutic context and objectives. One skilled in the art will appreciate that the appropriate dosage levels for treatment, according to certain embodiments, will thus vary depending, in part, upon the molecule delivered, the indication for which the pharmaceutical composition is being used, the route of administration, and the size (body weight, body surface or organ size) and/or condition (the age and general health) of the patient. A clinician may titer the dosage and modify the route of administration to obtain the optimal therapeutic effect.

The dosing frequency will depend upon the pharmacokinetic parameters of a therapeutic compound as described herein in the formulation. For example, a clinician administers the composition until a dosage is reached that achieves the desired effect. The composition may therefore be administered as a single dose, or as two or more doses (which may or may not contain the same amount of the desired molecule) over time, or as a continuous infusion via an implantation device or catheter. Further refinement of the appropriate dosage is routinely made by those of ordinary skill in the art and is within the ambit of tasks routinely performed by them. Appropriate dosages may be ascertained through use of appropriate dose-response data.

Administration routes for the pharmaceutical compositions of the invention include orally, through injection by intravenous, intraperitoneal, intracerebral (intra-parenchymal), intracerebroventricular, intramuscular, intra-ocular, intraarterial, intraportal, subcutaneous, or intralesional routes; by sustained release systems or by implantation devices. The pharmaceutical compositions may be administered by bolus injection or continuously by infusion, or by implantation device. Pharmaceutical compositions of the invention for use in systemic injections would allow effective delivery of the therapeutic compounds across the blood-brain barrier to a subject's CNS. The pharmaceutical composition also can be administered locally via implantation of a membrane, sponge or another appropriate material onto which the desired molecule has been absorbed or encapsulated. Where an implantation device is used, the device may be implanted into any suitable tissue or organ, and delivery of the desired molecule may be via diffusion, timed-release bolus, or continuous administration.

The Examples, which follow, are illustrative of specific embodiments of the invention, and various uses thereof. They are set forth for explanatory purposes only, and are not to be taken as limiting the invention.

### EXAMPLES

### Example 1

### APP Knockout Mice Exhibited Reduced Neural Stem Cell Proliferation and Reduced Neural Differentiation

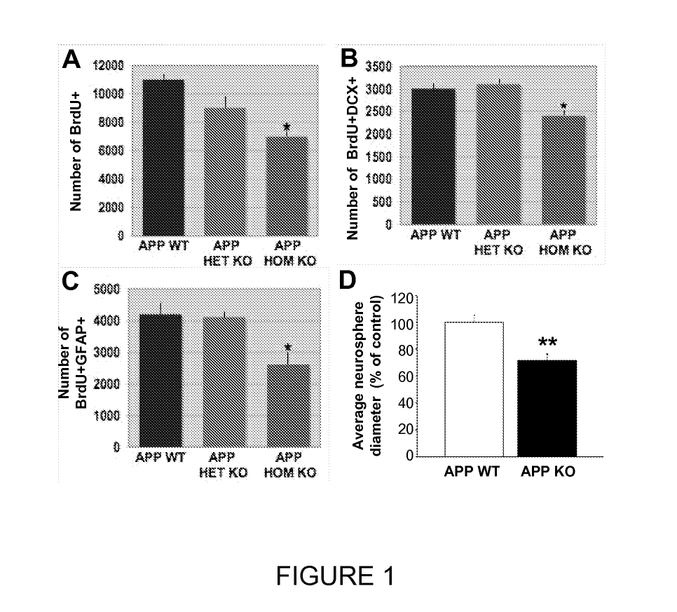

To examine whether lack of APP expression altered proliferation of NSCs in the adult brain, six-month-old knockout mice homozygous (APPKOHOM) or heterozygous (APPKOHET) for APP knockout and APP wild type (APPWT) mice were injected with BrdU (100 mg/kg) for three days, twice a day and then sacrificed (N=6). Sagittal brain sections (50 μm) of the mice were immunostained with antibodies specific for BrdU (which labels proliferating cells; the antibody can be obtained commercially from for example Fitzgerald, Concord, Mass.), an early neuronal marker doublecortin (DCX, the antibody can be obtained commercially from for example Abgent, catalog No. Ap2768a, San Diego, Calif.), and an astrocyte marker glial fibrillary acidic protein (GFAP, the antibody can be obtained commercially from for example Abcam, catalog No. ab929). Stereological analysis of the number of BrdU-labeled NSC in brain sections revealed a significant decrease in the number of proliferating neural progenitor cells (BrdU+; FIG. 1A), a decrease in newly-differentiating neurons (BrdU+DCX+; FIG. 1B) and a decrease in newly-differentiating astrocytes (BrdU+GFAP+; FIG. 1C) in the subventricular zone (SVZ) of APPKOHOM as compared to APPWT. No significant differences were observed in the percentages of newly differentiated neurons or astrocytes over all BrdU+ cells because the overall number of BrdU+ cells was reduced as a result of reduced proliferation (data not shown). No difference in the rate of NSC proliferation was detected in the subgranular layer (SGL) (data not shown). Clonogenic assays of neurospheres established by NSCs isolated from APPKOHOM and APPWT brains revealed a dramatic decrease in the size of neurospheres with APP knockout (FIG. 1D), suggesting reduced proliferation of neural progenitor cells isolated from APP knockout mice.

### Example 2

### The App Knockout Mice Expressed Reduced or Absent Levels of Secreted APP (sAPP)

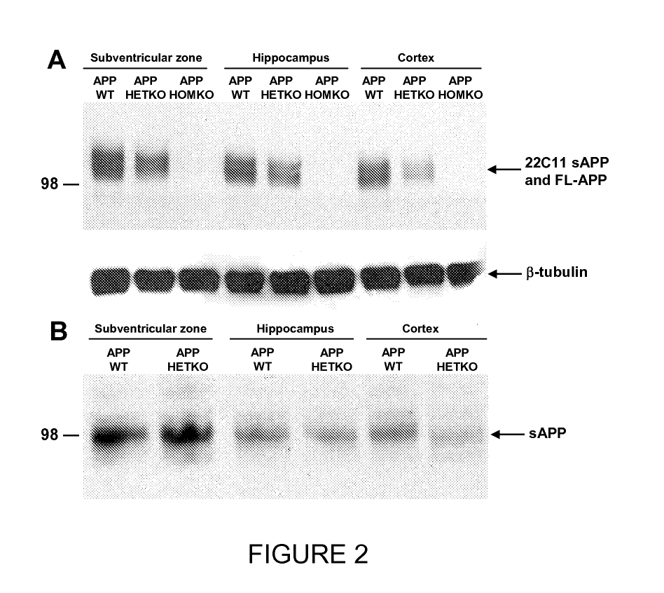

It has been reported that sAPP regulates proliferation of EGF-responsive neural progenitor cells in the SVZ (Caille et al., 2004, Development 131: 2173-81). Here, steady state levels of sAPP were examined in protein extracts of neurogenic areas, i.e., SVZ and hippocampus, and a normeurogenic area, i.e., cortex, from APP wild type and knockout mice by immunoblot analysis using an APP-specific monoclonal antibody (the 22C11 antibody, see Hilbich et al., 1993, J. Biol. Chem. 268:26571-26577) raised against the N-terminus of APP. Expression levels of full-length APP (FL-APP) and sAPP were significantly higher in protein extracts of SVZ compared to hippocampus and cortex (FIG. 2A). To discriminate between FL-APP and sAPP, immunodepletion was performed where FL-APP was depleted from protein extracts of the SVZ, hippocampus and cortex using a C-terminal APP-specific polyclonal antibody (the 369 antibody, see Kim et al. 2001, J. Biol. Chem. 276:43343-43350) that does not react with sAPP. Examination of the levels of sAPP after immunodepletion of the FL-APP was carried out using the 22C11 antibody by immunoblot analysis. The results revealed significantly higher levels of sAPP in the protein extracts of SVZ, as compared to the cortex of both wild type and APPKOHET mice (FIG. 2B). The higher levels of sAPP in the neurogenic niche SVZ as compared to the non-neurogenic area cortex indicated that sAPP is critical for regulating neural progenitor cells.

### Example 3

### Alpha-Secretase Inhibitor Reduced In Vitro Neural Stem Cell Proliferation

sAPP is the proteolytic product of α-secretase activity. The role for α-secretase in regulating proliferation of NSCs was examined. Neurosphere cultures were established from neural stem cells isolated from the SVZ of adult wild type mice. The neurospheres were dissociated to single cells, cultured in a 12-well plate (10,000 cells/well) and treated for 48 hours with GM6001, a broad-spectrum hydroxamic acid-based ADAM (A Disintegrin And Metalloprotease) inhibitor (Endres et al., 2005, FEBS J. 272:5808-20; Lemieux et al., 2007, J Biol. Chem. 282:14836-44). As a control, neurosphere cultures were treated with the inactive form of the inhibitor (GM6001NK). The compounds GM6001 and GM6001NK are obtainable from commercial sources such as Millipore (catalog No. CC1000, Billerica, Mass.) and Calbiochem (San Diego, Calif.). Thereafter, cells were pulse-labeled with 5 μM BrdU for 24 hours before fixation in 4% paraformaldehyde (PFA). The fixed cells were incubated with anti-BrdU antibodies and then HRP-conjugated secondary antibodies, followed by incubation with the TMD peroxidase substrate (Pierce Protein Research Products, Thermo Scientific, Rockford, Ill.). Signals were detected using a plate reader at 450-595 nm.

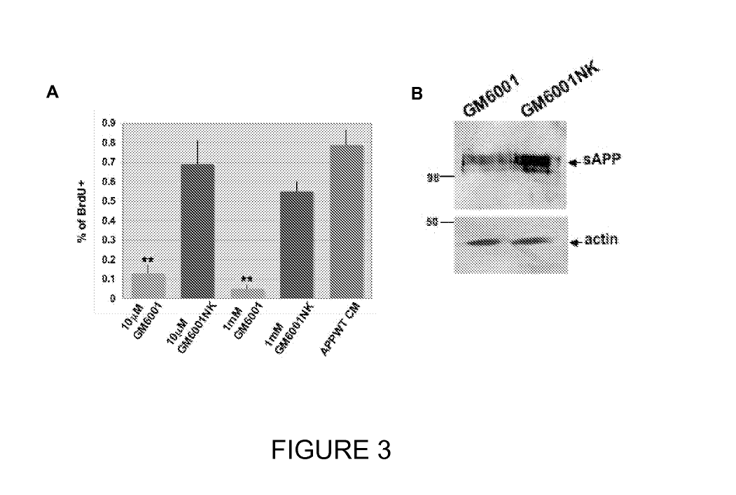

Results were obtained as percentages of BrdU positive cells in GM6001-treated culture over BrdU positive cells in the control culture treated with DMSO (FIG. 3A). Reduced BrdU immunoreactivity was observed in cells treated with GM6001 as compared to cells treated with its inactive analog Inhibition of alpha-secretase activity can affect sAPP levels. To examine the possibility that reduced proliferation was a result of reduced levels of sAPP, GM6001-treated cells were supplemented with conditioned media of neurospheres derived from APPWT mice, in which the α-secretase cleavage product sαAPP was present. The results showed recovery of BrdU immunoreactivity in GM6001-treated cells after supplemented with APPWT conditioned media (APPWT CM; FIG. 3A). The secretion levels of sAPP were also examined by immunoblot analysis in the conditioned media of GM6001- or GM6001NK-treated cells. As shown in FIG. 3B, levels of secreted sAPP were reduced in GM6001-treated NSC cultures.

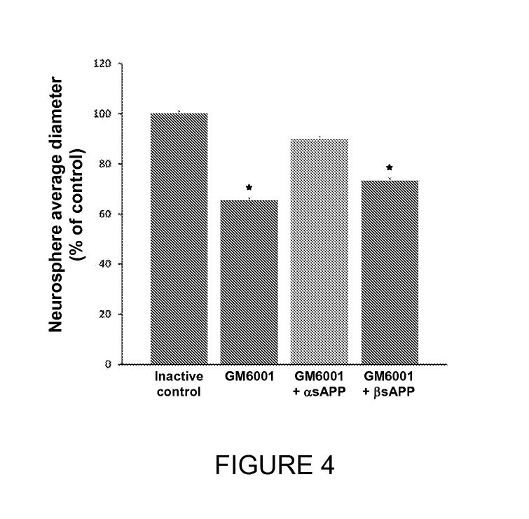

Both α- and β-secretases can cleave membrane-bound APP and release the secreted form of APP (sAPP). Proteolytic cleavage of APP by α-secretase releases one form of the secreted APP referred to as sαAPP, whereas cleavage of APP by β-secretase releases a C-terminally truncated sAPP referred to as sβAPP. The significance and difference in the functionality of these two forms of sAPP remains elusive. To distinguish the role of the α or β form of sAPP in promoting neural stem cell proliferation, neurospheres derived from adult mice neural stem cells were dissociated to single cells, plated for 8 days, treated with GM6001 or GM6001NK alone, treated with GM6001 and supplemented with conditioned media from neuroblastoma N2a cells expressing wild type APP, or treated with GM6001 and supplemented with conditioned media from 192 Swe N2a cells. Media conditioned by the neuroblastoma N2a was enriched in sαAPP as the wild type APP expressed in these cells was preferentially processed by α-secretase to produce sαAPP. The 192 Swe N2a conditioned medium was enriched in sβAPP as the 192 Swe N2a cells expressing the Swedish mutant form of APP that was preferentially cleaved by β-secretase. See Bogdanovic et al., 2001, Dement Geriatr. Congn. Discord. 12:364-70. As shown in FIG. 4, inhibition of α-secretase reduced the average diameter of neurospheres and this impairment was rescued by the addition of media containing sαAPP. However, media conditioned from 192 Swe N2a cells (the sβAPP-enriched conditioned medium) was unable to rescue the proliferative deficits as a result of reduced α-secretase activity.

### Example 4

### Generation of Lentiviral Vector Expressing Presenilin 1 (PS1) siRNA

In addition to α-secretase, APP is also a substrate for γ-secretase. The familial forms of the Alzheimer's disease (FAD) have been associated with mutations in APP, presenilin-1 (PS1) and presenilin-2 (PS2), the latter two being the proteolytic components of γ-secretase. Evidence has shown that neurogenesis is altered in the AD brain and in transgenic mice harboring mutant PS1. However, the mechanism underlying these alterations is largely unknown.

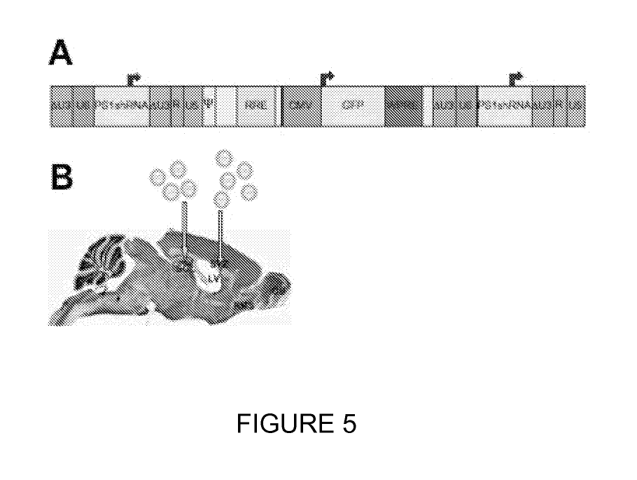

Mice that had the PS1 gene knocked out died in late embryogenesis. Thus, it has been difficult to generate PS1 knockout mice. To examine the role of PS1 in the proliferation, migration and maturation of neural progenitor cells in the adult mice brain, a third generation (self-inactivating) lentiviral vector system was selected for the construction of a lentiviral vector expressing PS1 siRNA. (Tiscornia et al., 2003, Proc. Natl. Acad. Sci. USA 100:1844-48; Brummelkamp et al., 2002, Science 296:550-553, epublication before print 2002 Mar. 21). The lentiviral vector provided in the instant application expressed a green fluorescent protein (GFP) marker to allow tracking of the targeted cells. The PS1 siRNA lentiviral vector also expressed a small-hairpin RNA (shRNA) from the 3′ remnant U3 sequence, which was processed in the cell to siRNA targeting PS1 RNA. See FIG. 5A. The shRNA expression cassette was generated by PCR amplification of the H1 promoter sequence with the addition of the shRNA sequences and a termination signal (TTTTT). The expression cassette sequence was inserted into the LTR region of the lentiviral vector (Tiscornia et al., 2003, supra), obtained from Dr. Robert Marr, Department of Neuroscience, The Rosalind Franklin University of Science and Medicine, North Chicago. The siRNA sequences targeting murine PS1 were designed based on a previously published sequence (5′AAGGCCCACTTCGTATGCTGG 3′ herein referred to as PS1 siRNA 1-1) (SEQ ID NO:17) with the aid of the algorithm S-fold (http://sfold.wadsworth.org/index.pl). See (Xie et al., 2004, J Biol Chem 279:34130-7).

The viral stocks were purified according to protocols established for preparing lentiviral vectors for gene transfer into the brain (Naldini et al., 1996, Science 272:263-267; Marr et al., 2003, J Neurosci 23:1992-1996; Hashimoto et al., 2004, Gene Ther 11:1713-1723; Hovatta et al., 2005, Nature 438:662-666; Singer et al., 2005, Nat Neurosci 8:1343-1349; Tiscornia et al., 2006, Nat Protoc 1:241-245). HEK-293T cells were transfected with recombinant lentiviral vectors and packaging plasmids as previously described (Tiscornia et al., 2006, supra). After transfection, the cell culture medium was changed to serum-free medium OPTIMEM® (Invitrogen, Carlsbad, Calif.). Transfected cell culture supernatant containing the packaged virus was then collected. Lentiviral vectors were purified by two rounds of ultracentrifugation at 50,000×g (the second centrifugation over a 20% sucrose cushion) (Tiscornia et al., 2006, supra). The final pellet was resuspended in sterile salt solution (Hank's Balanced Salt Solution) and any particles were removed by low speed centrifugation. The supernatant was used for intracranial injection. FIG. 5B illustrates the stereotaxic injection sites of the lentivirus, SGL and SVZ, as indicated by the arrows marked on the mouse brain section.

### Example 5

### Analysis of the Effects of PS1 siRNA Lentivirus in Injected Mouse Brain or Transduced Cells

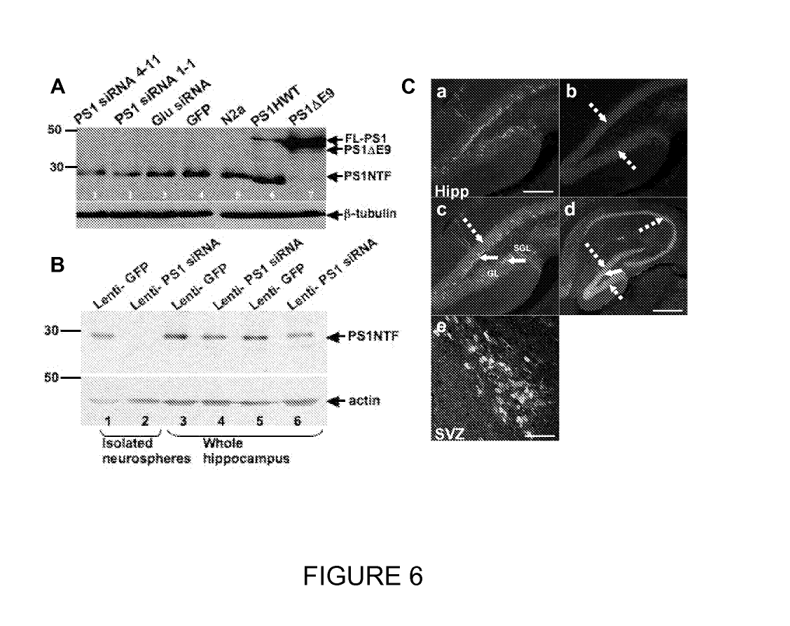

PS1 siRNA lentiviruses were verified by immunoblot analysis for the reduction of PS1 protein expression in N2a cells transduced with the lentiviral vector. N2a cells were transduced with the purified shRNA vector preparation followed by immunoblot analysis using anti-PS1 polyclonal antibody (Lazarov et al., 2005, J. Neurosci. 25: 2386-95.). Presenilins undergo cleavage in an alpha helical region of one of the cytoplasmic loops to produce a larger N-terminal (“PS1 NTF”) and a smaller C-terminal fragment which together form part of the functional protein. As shown in FIG. 6A, PS1 expression was reduced in N2a cells five days following transduction with lentiviral vectors expressing PS1 siRNA (lanes 1 and 2) as compared to N2a transduced with lentiviral vectors expressing GFP only (lane 4) or an irrelevant siRNA (Glu siRNA; lane 3). PS1 siRNA 4-11 (lane 1) refers to a different PS1 siRNA construct having the siRNA sequence of 5′ GGACCAACTTGCATTCCAT 3′ (SEQ ID NO:18) under the U6 promoter. The samples in lanes 6 and 7 were brain extracts of transgenic mice harboring PS1HWT (wild type human PS1; lane 6) and PS1ΔE9 (human PS1 with exon 9 deleted; lane 7), both of which served as positive controls for PS1 detection.

To examine knock down of PS1 expression in vivo, lentiviral vectors expressing GFP and PS1siRNA or GFP alone were stereotaxically injected unilaterally into the SVZ (subventricular zone) or hippocampus of C57/B16 mice (1 μl/site; 0.25 μl/minute) using the following coordinates: SVZ [coordinates (AP=+1.0 mm, ML=+1.0 mm, DV=−2.1 mm; AP=0 mm, ML=+1.25 mm, DV=−2.2 mm)]; DG [coordinates (AP=−2.0 mm, ML=+1.3 mm, DV=−2.0; AP=−3.0 mm, ML=+3 mm, DV=−3.0)]. Six weeks later, mice were sacrificed. Expression of PS1 in the SVZ and DG was examined by immunoblot analysis (FIG. 6B). As expected, PS1 expression was dramatically reduced in neurospheres isolated from the DG of mice ipsilaterally injected with lentiviral vectors expressing siRNA for PS1 targeting (FIG. 6B compare lanes 1 and 2), and modestly reduced in protein extract of whole DG (FIG. 6B compare lanes 3 and 5 versus 4 and 6).

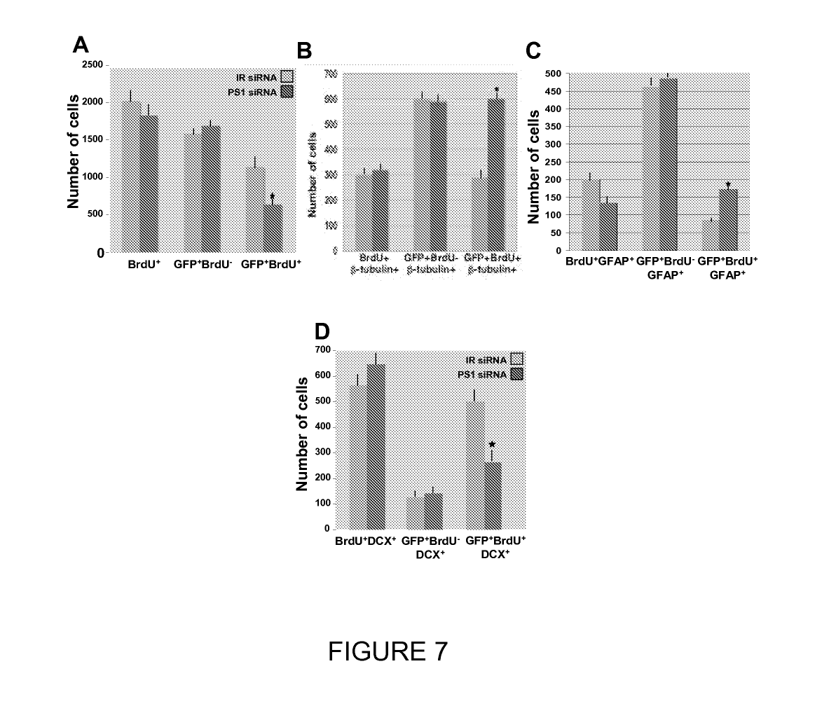

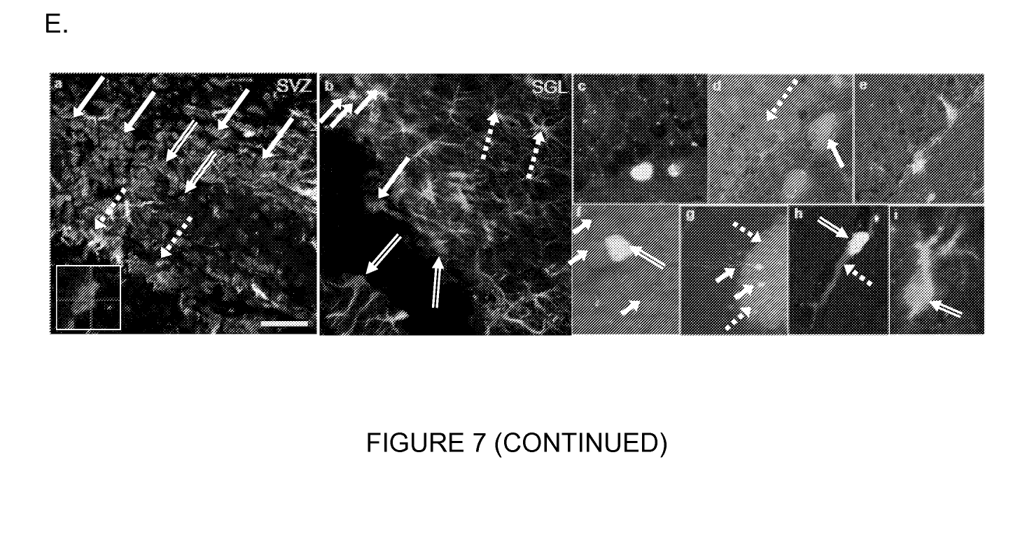

Brain sections were examined for the presence of GFP+ cells (FIG. 6C). GFP+ cells were detected in the dentate gyms (DG) (FIG. 6C panels a-d) and SVZ (FIG. 6C panel e) of adult mice six weeks after lentiviral vectors injection. In the DG, the vast majority of GFP+ cells could be detected in the SGL (subgranule layer) (FIG. 6C panels a,c,d). Some GFP+ cells migrated to the GL (granule layer) and even extended processes towards the outer molecular layer of the DG (FIG. 6C panel a), as previously shown (van Praag et al., 2002, Nature 415:1030-1034). Merged images of immunostaining of a mature neuronal marker NeuN with the immunostaining of GFP shown in the DG (panel c) and in the whole hippocampus (panel d) revealed that GFP+ cells in the SGL were NeuN-, suggesting that these were newly-formed cells (FIG. 6C compare panels c and d). This result is also demonstrated in FIG. 6C panels a and b, where GFP+ cells in the SGL (panel a) were not immunoreactive for the mature neuronal marker NeuN (panel b), suggesting that these GFP+ cells were yet differentiated into neurons. However, when the GFP+ cells migrated to the granule layer, the GFP+ cells became positive of NeuN staining (see FIG. 7E panel f as described below).

In separate experiments, mice were further injected with BrdU (100 mg/kg) twice a day for three days after stereotaxic injection of lentiviral vectors for six weeks to determine whether the GFP+ cells were proliferating. As shown in FIG. 6C, panel e, the vast majority of GFP+ cells in SVZ were also BrdU+. This was observed in both PS1 siRNA lentivirus-injected and IR siRNA-lentivirus-injected mice.

### Example 6

### Transduction of PS1 siRNA Lentiviral Vectors Reduced the Number of Proliferating NSCs in the Dentate Gyms and Increased Neural Differentiation

To analyze and quantify the effects of PS1 siRNA on neural stem cell proliferation and differentiation, six weeks after stereotaxic injection of lentiviral vectors, mice were pulse-labeled with BrdU (100 mg/kg) twice a day for three days and then sacrificed. Stereological analysis of the number of immunolabeled GFP+BrdU+, GFP+BrdU+β-tubulin+ and GFP+BrdU+GFAP+ cells in the dentate gyms in brain sections of these mice was performed. The number of GFP+ cells was comparable in mice injected with lentiviral vectors expressing an irrelevant (IR) siRNA and in mice injected with vectors expressing PS1 siRNA (N=6; FIG. 7A). In mice injected with a lentiviral vector expressing IR siRNA, about 50% of GFP+ cells were BrdU+, suggesting that 50% of the GFP+ cells underwent proliferation during the last three days of the animal's life. A significant reduction in the number of GFP+BrdU+ was observed in brain sections of mice expressing siRNA for PS1 targeting (N=6; FIG. 7A). To examine whether reduction in the rate of proliferation in cells expressing siRNA for PS1 targeting was accompanied by increased differentiation, the number of newly differentiated neurons (GFP+BrdU+β-tubulin+) and newly differentiated astrocytes (GFP+BrdU+GFAP+) was quantified by stereological analysis. The results showed a significant increase in the number of newly differentiated neurons and astrocytes in brain sections of mice six weeks after injection of lentiviral vectors expressing siRNA for PS1 targeting (N=6; FIGS. 7B-C). β-tubulin is a late neural marker. Under the same experimental conditions, the number of GFP+ cells that expressed an early neural marker DCX was decreased in mice transduced with the lentiviral vector expressing PS1 siRNA as compared to control (N=4; FIG. 7D).

FIG. 7E shows representative confocal images of cells detected in the SGL and SVZ of mouse transduced with PS1 siRNA lentiviral vector by immunofluorescence staining. The distribution of NSCs is shown by detecting BrdU+ cells in SVZ (panel a) and SGL (panel b) in an adult mouse. The BrdU+ cells (marked by single arrows) demonstrated neural progenitor cells in the SVZ and SGL. The images in panels c to i were immunostaining of brain sections from mice injected with the PS1 siRNA lentiviral vector. Increased neural differentiation was not readily seen in the SGL three weeks after transduction of PS1 siRNA lentiviral vector. As shown in panel d, the GFP+ cells were NeuN negative. After six weeks post transduction, GFP+BrdU+ cells at the SGL that extended processes towards the granule layer of the DG were detected (panel e). Further, GFP+NeuN+ cell incorporated in the granule layer of the DG was detected as shown in panel f (as indicated by the double arrow in panel f). Neural differentiation was also evidenced in panel g where GFP+ cells migrated to the granule cell layer of the DG and extended processes towards the molecule cell layer of the DG (as indicated by the single arrows in panel g). GFP+ cells immunopositive for DCX and GFAP were also detected as shown in panel h and panel i, respectively.

### Example 7

### Neurospheres Treated with γ-Secretase Inhibitor Exhibited Reduced Proliferation and Increased Neural Differentiation

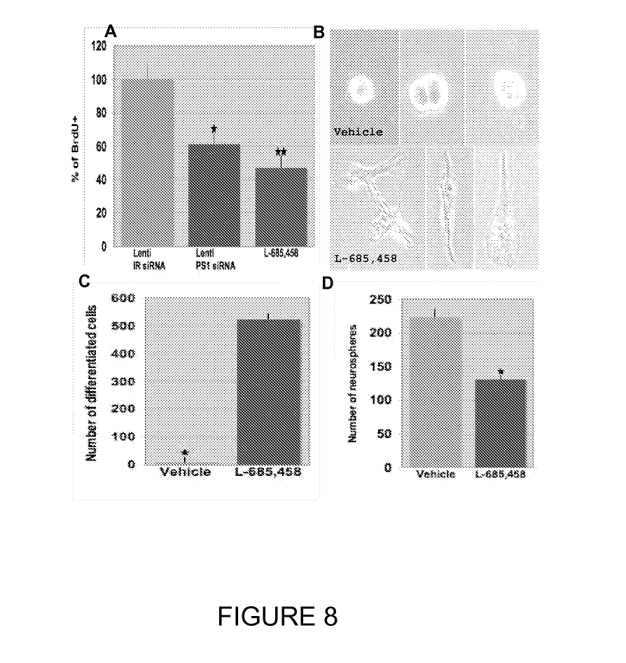

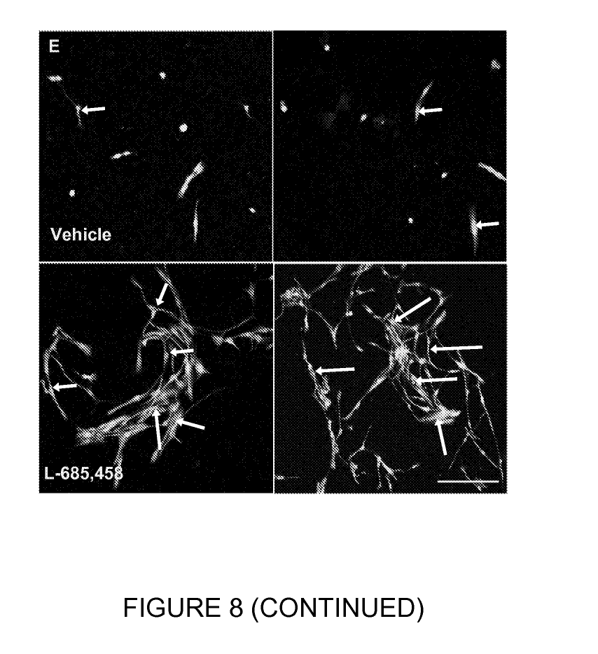

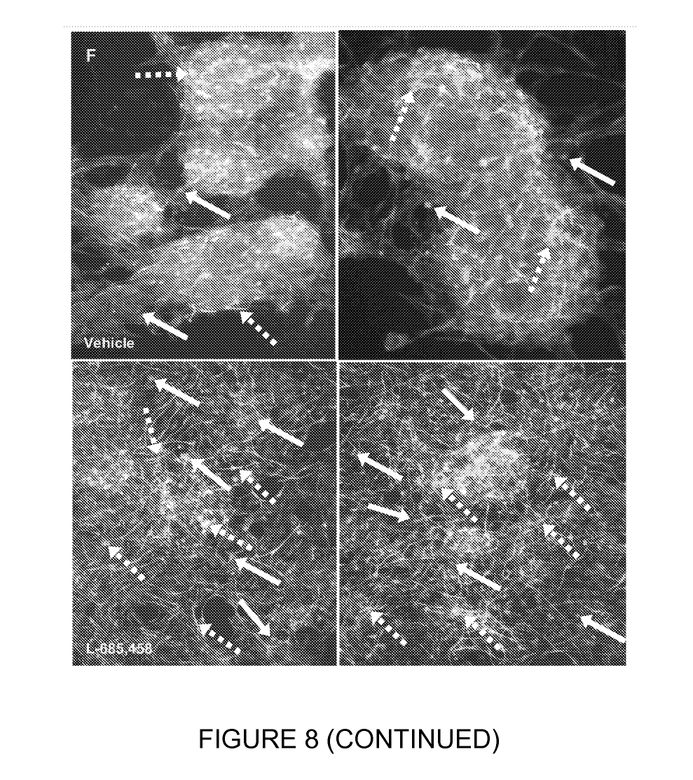

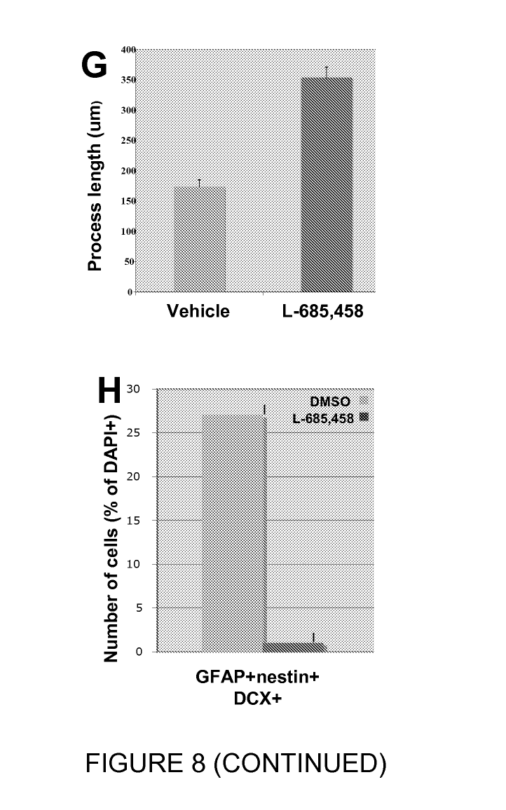

To further establish that reduced expression of PS1 decreased neural stem cell proliferation and induced neural stem cell differentiation, neurospheres established as described above from the SVZ of adult mice were subject to a proliferation assay following the treatment with the γ-secretase inhibitor L-685,458 (1 μM for 24 hours) (Sigma, St. Louis, Mo.). As a comparison, neurospheres were transduced with lentiviral vectors expressing either IR siRNA or PS1 siRNA. The proliferation assay was performed as follows: lentivirally-transduced neurospheres or γ-secretase inhibitor-treated neurospheres were singly dissociated and cultured (10,000 cells/well) with BrdU for 48 hours. BrdU-labeled cells were fixed in 4% PFA and immunolabeled with anti-BrdU antibodies followed by HRP-conjugated secondary antibodies. Thereafter, the cells were incubated with the TMD peroxidase substrate and read on a plate reader at 450-595 nm. Reduced BrdU+ immunoreactivity was observed in cells treated with γ-secretase inhibitor, as well as cells transduced with lentiviral vector expressing PS1 siRNA (FIG. 8A). Results were presented as a percentage of DMSO-treated NSCs.

The effects of γ-secretase inhibitor L-685,458 on neural stem cell differentiation was analyzed. Neural stem cells were allowed to differentiate when cultured on glass coverslips coated with 10 μg/mL poly-L-ornithine (Sigma, St. Louis, Mo.) and 5-10 μg/mL laminin (Sigma) in media with 5% fetal bovine serum without EGF and FGF. As shown in FIGS. 8B-8F, γ-secretase inhibitor L-685,458 induced neural progenitor cell differentiation in the neural differentiation culture medium as compared to cells treated with DMSO. Phase contrast images of the cultured cells showed neural stem cells differentiating following treatment with L-685,458 (FIG. 8B, lower panels), while cells maintained their undifferentiated neurosphere state in the vehicle-treated group (FIG. 8B, upper panels). The increased number of differentiated cells after a two-day treatment of L-685,458 as compared to control is shown in FIG. 8C; and the reduced number of neurospheres formed from singly-dissociated neurosphere cells following a two-day treatment with L-685,458 as compared with control is shown in FIG. 8D.

γ-secretase inhibitor L-685,458-induced differentiation was further analyzed by immunostaining using antibodies to the astrocyte marker GFAP, and the early neural marker nestin. Positive staining of GFAP and morphology changes to more differentiated cells was detected in L-685,458-treated cells indicating increased differentiation. Scale bar=75 μM (FIG. 8E, representative GFAP staining is marked with single arrows). In fact, the results as shown in FIG. 8H demonstrated that after a two-day treatment of L-685,458, the number of GFAP+Nestin+DCX+ cells, i.e., neural progenitor cells expressing early markers of neuronal differentiation, was dramatically decreased as compared to control.

The length of the processes developed from the middle of the soma to the axon tip of GFAP positive cells after two days of L-685,458 treatment was analyzed and the results are shown in FIG. 8G. Twenty four hours after culturing whole neurospheres on laminin, vehicle-treated cultures remained mostly as neurospheres with slight cell differentiation (FIG. 8F, top panels). On the other hand, L-685,458 inhibitor-treated cultures exhibited extensive differentiation and only a portion of the cells remained as neurospheres (FIG. 8F, bottom panels). Neural differentiation was confirmed by the increased immunostaining for both β-tubulin and GFAP (FIG. 8F, bottom panels).

### Example 8

### Neurospheres Transduced with PS1 siRNA Expressing Lentiviral Vectors Exhibited Reduced Proliferation and Increased Neural Differentiation

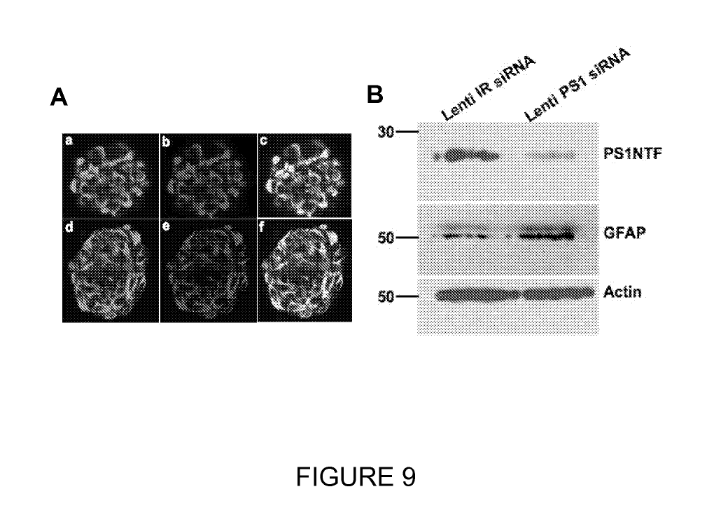

As shown in FIG. 8A, inhibition of PS1 by either PS1 siRNA or γ-secretase inhibitor L-685,458 reduced neural stem cell proliferation. The effects of PS1 siRNA on neural differentiation of neurospheres were examined in this experiment. Neurosphere cultures were established from neural stem cells isolated from the subventricular zone of adult mice as described above. Neurospheres were singly dissociated and transduced with five transducing units/ml (i.e., multiplicity of infection, i.e., MOI, of 5) of lentiviral vectors expressing either an IR siRNA or PS1 siRNA. The medium was replaced 24 hours later. GFP+ neurospheres were detected three days after transduction (FIG. 9A, panels a and d). Nestin positive neurospheres were detected as shown in FIG. 9A, panels b and e, and the GFP+Nestin+ double staining was detected in the merged images in panels c and f. Further, in protein extract of neurosphere culture expressing PS1 siRNA, PS1 expression was reduced as detected by immunoblot analysis, while GFAP expression was increased (FIG. 9B).

It should be understood that the foregoing disclosure emphasizes certain specific embodiments of the invention and that all modifications or alternatives equivalent thereto are within the spirit and scope of the invention as set forth in the appended claims.

1-63. (canceled)

64. A method of inducing differentiation in a neural stem cell comprising the step of decreasing γ-secretase activity in a neural stem cell, wherein the γ-secretase activity is decreased by a γ-secretase inhibitor, and wherein the γ-secretase inhibitor does not affect or inhibit Notch activity.

65. The method of claim 64, wherein the neural stem cell is present in a subject's central nervous system (CNS), and the induced differentiation in the neural stem cell promotes neurogenesis in the subject's CNS.

66. The method of claim 64, wherein the γ-secretase inhibitor comprises a presenilin-1 (ps-1) siRNA.

67. The method of claim 66, wherein the ps-1 siRNA comprises the nucleotide sequence of SEQ ID NO:17 or SEQ ID NO:18.

68. A method of treating a neurodegenerative disease in a subject comprising the step of decreasing γ-secretase activity in a subject's CNS, wherein the γ-secretase inhibitor does not affect or inhibit Notch activity, and wherein the decreased γ-secretase activity results in increased neural differentiation in the subject's CNS.

69. The method of claim 68, wherein the increased neural differentiation in the subject's CNS promotes neurogenesis in the subject's CNS.

70. The method of claim 68, wherein the γ-secretase activity is decreased by a γ-secretase inhibitor in a neural stem cell in the subject's CNS.

71. The method of claim 70, wherein the γ-secretase inhibitor comprises a presenilin-1 (ps-1) siRNA.

72. The method of claim 71, wherein the ps-1 siRNA comprises the nucleotide sequence of SEQ ID NO:17 or SEQ ID NO:18.

73. The method of claim 68, wherein the neurodegenerative disease is an aging-related neurodegenerative disease.

74. The method of claim 73, wherein the aging-related neurodegenerative disease is Alzheimer's Disease, dementia, Parkinson's Disease, Huntington's disease, amyotrophic lateral sclerosis (ALS), or mild cognitive impairment (MCI).

75. The method of claim 74, wherein the subject is a human.

76. A method of promoting neural stem cell proliferation comprising the step of increasing α-secretase activity in a neural stem cell.

77. The method of claim 76, wherein the α-secretase activity is increased by providing exogenous expression of a gene that possesses α-secretase activity in the neural stem cell.

78. The method of claim 77, wherein the gene is ADAM9, ADAM10 or ADAM17.

79. The method of claim 78, wherein the gene is ADAM10.

80. The method of claim 76, wherein the increased α-secretase activity increases the levels of secreted amyloid precursor protein (sαAPP) in the neural stem cell.

81. The method of claim 76 wherein the neural stem cell is present in a subject's CNS, and wherein the increased neural stem cell proliferation promotes neurogenesis in the subject's CNS.

82. A method of promoting neural stem cell proliferation comprising the step of contacting a neural stem cell with a cell that expresses a protein having α-secretase activity.

83. The method of claim 82, wherein the cell that contacts the neural stem cell expresses an exogenous gene that possesses α-secretase activity.

84. The method of claim 83, wherein the gene is ADAM9, ADAM10 or ADAM17.

85. The method of claim 84, wherein the gene is ADAM10.

86. The method of claim 82, wherein the neural stem cell is present in a subject's CNS and the increased neural stem cell proliferation promotes neurogenesis in the subject's CNS.

87. A method of treating a neurodegenerative disease in a subject comprising the step of increasing α-secretase activity in the subject's CNS wherein the increased α-secretase activity results in increased neural stem cell proliferation in the subject's CNS.

88. The method of claim 87, wherein the α-secretase activity is increased by providing exogenous expression of a gene that possesses α-secretase activity in the subject's CNS.

89. The method of claim 88, wherein the α-secretase activity is increased by providing exogenous expression of the gene that possesses α-secretase activity in a neural stem cell in the subject's CNS.

90. The method of claim 88, wherein the α-secretase activity is increased by contacting the subject's CNS with a cell that expresses a protein having α-secretase activity.

91. The method of claim 90, wherein the cell that contacts the subject's CNS is a cell derived from the subject.

92. The method of claim 90 wherein the cell that contacts the subject's CNS expresses an exogenous gene that possesses α-secretase activity.

93. The method of claim 92, wherein the gene is ADAM9, ADAM10 or ADAM17.

94. The method of claim 93, wherein the gene is ADAM10.

95. The method of claim 87, wherein the neurodegenerative disease is an aging-related neurodegenerative disease.

96. The method of claim 95, wherein the aging-related neurodegenerative disease is Alzheimer's Disease, dementia, Parkinson's Disease, Huntington's disease, amyotrophic lateral sclerosis (ALS), or mild cognitive impairment (MCI).

97. The method of claim 96, wherein the subject is a human.

98. A method of promoting neurogenesis in a subject comprising contacting the subject's CNS with the α form of the secreted amyloid precursor protein (sαAPP).

99. The method of claim 98, wherein the subject is suffering from a neurodegenerative disease.

100. The method of claim 99, wherein the neurodegenerative disease is an aging-related neurodegenerative disease.

101. The method of claim 100, wherein the aging-related neurodegenerative disease is Alzheimer's Disease, dementia, Parkinson's Disease, Huntington's disease, amyotrophic lateral sclerosis (ALS), or mild cognitive impairment (MCI).

102. The method of claim 99, wherein the subject is a human.


SEQUENCE LISTING (22 sequences)

SEQ ID NO: 1 - DNA, 3836 bp, Homo sapiens
  CDS (39)..(2006)
cgaccgagtgctgagaggaacctgcggaatcggccgagatggggtctggcgcgcgctttccctcggggacccttcgtgtccggtggttgctgttgcttggcctggtgggcccagtcctcggtgcggcgcggccaggctttcaacagacctcacatctttcttcttatgaaattataactccttggagattaactagagaaagaagagaagcccctaggccctattcaaaacaagtatcttatgttattcaggctgaaggaaaagagcatattattcacttggaaaggaacaaagaccttttgcctgaagattttgtggtttatacttacaacaaggaagggactttaatcactgaccatcccaatatacagaatcattgtcattatcggggctatgtggagggagttcataattcatccattgctcttagcgactgttttggactcagaggattgctgcatttagagaatgcgagttatgggattgaacccctgcagaacagctctcattttgagcacatcatttatcgaatggatgatgtctacaaagagcctctgaaatgtggagtttccaacaaggatatagagaaagaaactgcaaaggatgaagaggaagagcctcccagcatgactcagctacttcgaagaagaagagctgtcttgccacagacccggtatgtggagctgttcattgtcgtagacaaggaaaggtatgacatgatgggaagaaatcagactgctgtgagagaagagatgattctcctggcaaactacttggatagtatgtatattatgttaaatattcgaattgtgctagttggactggagatttggaccaatggaaacctgatcaacatagttgggggtgctggtgatgtgctggggaacttcgtgcagtggcgggaaaagtttcttatcacacgtcggagacatgacagtgcacagctagttctaaagaaaggttttggtggaactgcaggaatggcatttgtgggaacagtgtgttcaaggagccacgcaggcgggattaatgtgtttggacaaatcactgtggagacatttgcttccattgttgctcatgaattgggtcataatcttggaatgaatcacgatgatgggagagattgttcctgtggagcaaagagctgcatcatgaattcaggagcatcgggttccagaaactttagcagttgcagtgcagaggactttgagaagttaactttaaataaaggaggaaactgccttcttaatattccaaagcctgatgaagcctatagtgctccctcctgtggtaataagttggtggacgctggggaagagtgtgactgtggtactccaaaggaatgtgaattggacccttgctgcgaaggaagtacctgtaagcttaaatcatttgctgagtgtgcatatggtgactgttgtaaagactgtcggttccttccaggaggtactttatgccgaggaaaaaccagtgagtgtgatgttccagagtactgcaatggttcttctcagttctgtcagccagatgtttttattcagaatggatatccttgccagaataacaaagcctattgctacaacggcatgtgccagtattatgatgctcaatgtcaagtcatctttggctcaaaagccaaggctgcccccaaagattgtttcattgaagtgaattctaaaggtgacagatttggcaattgtggtttctctggcaatgaatacaagaagtgtgccactgggaatgctttgtgtggaaagcttcagtgtgagaatgtacaagagatacctgtatttggaattgtgcctgctattattcaaacgcctagtcgaggcaccaaatgttggggtgtggatttccagctaggatcagatgttccagatcctgggatggttaacgaaggcacaaaatgtggtgctggaaagatctgtagaaacttccagtgtgtagatgcttctgttctgaattatgactgtgatgttcagaaaaagtgtcatggacatgggaaatgaatactgcattgagggacggacttctggtcttcttcttcctaattgttccccttattgtctgtgctatttttatcttcatcaagagggatcaactgtggagaagctacttcagaaagaagagatcacaaacatatgagtcagatggcaaaaatcaagcaaacccttctagacagccggggagtgttcctcgacatgtttctccagtgacacctcccagagaagttcctatatatgcaaacagatttgcagtaccaacctatgcagccaagcaacctcagcagttcccatcaaggccacctccaccacaaccgaaagtatcatctcagggaaacttaattcctgcccgtcctgctcctgcacctcctttatatagttccctcacttgatttttttaaccttctttttgcaaatgtcttcagggaactgagctaatactttttttttttcttgatgttttcttgaaaagcctttctgttgcaactatgaatgaaaacaaaacaccacaaaacagacttcactaacacagaaaaacagaaactgagtgtgagagttgtgaaatacaaggaaatgcagtaaagccagggaatttacaataacatttccgtttccatcattgaataagtcttattcagtcatcggtgaggttaatgcactaatcatggattttttgaacatgttattgcagtgattctcaaattaactgtattggtgtaagatttttgtcattaagtgtttaagtgttattctgaattttctaccttagttatcattaatgtagttcctcattgaacatgtgataatctaatacctgtgaaaactgactaatcagctgccaataatatctaatatttttcatcatgcacgaattaataatcatcatactctagaatcttgtctgtcactcactacatgaataagcaaatattgtcttcaaaagaatgcacaagaaccacaattaagatgtcatattattttgaaagtacaaaatatactaaaagagtgtgtgtgtattcacgcagttactcgcttccatttttatgacctttcaactataggtaataactcttagagaaattaatttaatattagaatttctattatgaatcatgtgaaagcatgacattcgttcacaatagcactattttaaataaattataagctttaaggtacgaagtatttaatagatctaatcaaatatgttgattcatggctataataaagcaggagcaattataaaatcttcaatcaattgaacttttacaaaaccacttgagaatttcatgagcactttaaaatctgaactttcaaagcttgctattaaatcatttagaatgtttacatttactaaggtgtgctgggtcatgtaaaatattagacactaatattttcatagaaattaggctggagaaagaaggaagaaatggttttcttaaatacctacaaaaaagttactgtggtatctatgagttatcatcttagctgtgttaaaaatgaatttttactatggcagatatggtatggatcgtaaaattttaagcactaaaaattttttcataacctttcataataaagtttaataataggtttattaactgaatttcattagttttttaaaagtgtttttggtttgtgtatatatacatatacaaatacaacatttacaataaataaaatacttgaaattctcttttgtgtctcctagtagcttcctactcaactatttataatctcattaattaaaaagttataattttagataaaaattctagtcaaatttttacagatattatctcactaattttcagacttttgccaaagtgtgcacaatgg

SEQ ID NO: 2 - protein, 655 aa, Homo sapiens
MGSGARFPSGTLRVRWLLLLGLVGPVLGAARPGFQQTSHLSSYEIITPWRLTRERREAPRPYSKQVSYVIQAEGKEHIIHLERNKDLLPEDFVVYTYNKEGTLITDHPNIQNHCHYRGYVEGVHNSSIALSDCFGLRGLLHLENASYGIEPLQNSSHFEHIIYRMDDVYKEPLKCGVSNKDIEKETAKDEEEEPPSMTQLLRRRRAVLPQTRYVELFIVVDKERYDMMGRNQTAVREEMILLANYLDSMYIMLNIRIVLVGLEIWTNGNLINIVGGAGDVLGNFVQWREKFLITRRRHDSAQLVLKKGFGGTAGMAFVGTVCSRSHAGGINVFGQITVETFASIVAHELGHNLGMNHDDGRDCSCGAKSCIMNSGASGSRNFSSCSAEDFEKLTLNKGGNCLLNIPKPDEAYSAPSCGNKLVDAGEECDCGTPKECELDPCCEGSTCKLKSFAECAYGDCCKDCRFLPGGTLCRGKTSECDVPEYCNGSSQFCQPDVFIQNGYPCQNNKAYCYNGMCQYYDAQCQVIFGSKAKAAPKDCFIEVNSKGDRFGNCGFSGNEYKKCATGNALCGKLQCENVQEIPVFGIVPAIIQTPSRGTKCWGVDFQLGSDVPDPGMVNEGTKCGAGKICRNFQCVDASVLNYDCDVQKKCHGHGK

SEQ ID NO: 3 - DNA, 3881 bp, Mus musculus
  CDS (20)..(2557)
acctgccgaagccctcgctatggggccgcgcgcgctctcgccccttgcctctctgcgactaaggtggctgctggcgtgtggcttgctgggcccagtcctcgaggccgggcgaccagacttggaacagactgtccatctttcttcttatgaaattattactccttggagattaactagagaaagaagggaagctctggggcccagttcacagcagatctcttacgtcatccaggcccaaggaaaacagcatattattcacttggaaagaaacacagaccttttacctaatgattttgtagtttacacctacgacaaggaaggctccctactctctgaccatcccaacgtacagagccattgtcactatcgaggctatgtggagggagtgcagaattccgcggttgctgtgagcgcctgctttggactcagaggcttgctgcatttggagaatgccagttttggaattgaacctctgcacaacagctcacactttgagcacatattttaccccatggatggcatccaccaggagcctctgagatgtggagtctctaacagggacacagagaaggaaggcacacagggggatgaggaggagcatccgagtgtcactcagctgctgcgcagaagaagagctgttctaccacagacccgctatgtggagctgttcattgttgtagacaaggaaaggtacgacatgatgggacggaaccagactgctgtgagagaagagatgattcgcttagcaaactacctggatagcatgtacatcatgttaaacattcgaattgtgctggttggactagaaatttggacagacagaaatcctatcaatataattggaggagctggagatgtgctgggcaactttgttcagtggcgggaaaagttccttataactcgtcggagacacgacagtgcacagttggttttgaagaaaggctttggtggaactgcaggaatggcgtttgtaggaacagtatgttcaaggagccacgcaggtgggatcaatgtgtttgggcaaatcactgtggagacatttgcatccattgttgctcatgaattggggcataaccttggaatgaatcatgatgatgggagagagtgtttctgtggagcaaagagctgtatcatgaattcaggagcatccgggtccagaaactttagcagttgcagtgcggaggactttgagaagttaacgttgaataagggaggaagctgcctgcttaacatcccgaagcctgacgaagcctacagcgcgccctcctgtggtaataagctggtggaccctggagaggagtgtgactgcggcacagcgaaggagtgtgaggtggacccatgctgtgaaggaagcacttgtaagctcaagtcatttgctgagtgtgcatatggcgactgttgtaaagattgccagttccttccaggaggctccatgtgcagagggaagaccagtgagtgtgatgttcctgagtactgcaacggttcctctcagttctgcccgccagatgtcttcattcagaatggatatccttgccagaacagcaaagcctactgctacaatggcatgtgccaatattatgacgcgcagtgtcaggtcatctttggttcaaaggctaaggctgccccaagagattgcttcattgaagtcaattctaaaggtgacagatttggcaactgtggtttctccggcagtgagtacaagaagtgtgccactgggaacgcgctgtgtggaaagcttcaatgcgagaatgtacaggacatgccggtgtttggaatagtaccagctatcattcagacacccagtcgaggcaccaaatgctggggtgtggatttccagcttggttccgacgttccagacccagggatggtgaatgaaggcaccaaatgtgatgctggcaagatttgcaggaattttcagtgtgtaaatgcttctgtcctgaattatgactgtgacattcagggaaaatgtcatggccatggggtatgtaacagcaataagaattgtcactgtgaagatggctgggctcccccacactgtgacaccaaaggatatggaggaagcgtggacagcgggccgacgtataatgcaaagagcacagcactgagggacgggcttctggtcttcttcttcctaatcgtcccccttgttgcggctgccattttcctctttatcaagagagatgaactacggaaaaccttcaggaagaagagatcacaaatgtcagatggcagaaatcaagcaaacgtctctagacagccaggagatcctagtatctccagaccaccagggggcccaaatgtctccagaccaccagggggcccaggtgtctccagaccaccagggggcccaggtgtctccagaccaccagggggcccaggtgtctccagaccgccacctgggcatggaaacagattcccagtaccaacctacgccgccaagcagcctgcgcagttcccgtcaaggccacctccaccacaaccgaaaatatcttctcagggaaacttgattccggctcggcccgctcctgcacctcctttatatagctccctcacctgatagtagaatattagaatcttattttttaaatgtcttcagggaactgagcaaatgtttgttgtttttttttttcctgatgttttcttgaaaagcctttctcttccaaccatgaatgaacacaaaccaccacaaaacaagctttattaacacaggagcctagtggggattgcgaaacacaggaatgtgcaggcgctccggggggtgtaaagtgaacgtttccatcgttagaatgttttctctggccatttgtggatttaatgcacttgacgtggattaagttattctgagcatgttactgtaatgattctcaaattaactgtattagtgtaagctttgtcactatgcgctaaacgtaatcctgactttttgaccccagttaccattaatagtttctggttgaccatttgaatatgtattaacttaggaagactaattgccaataacgtctgcattttcatcttgcatggattaacagccatttatatggacttatgtctcttaatgcacaaagaagcagatatctcgaaggagcttacacaagaaccacaattactagatcatgatatacttggaaagtgtgaaatatggtgtgtactcagttattggcttccattttttatgatctttcaactataacaattatgatagaaatcgatttaacacaatcagttatgggcttccattttcaaatatcttttcaactgtaatgattatgacaggaactgattcaactctcaattttctttatgcatcatggtaaagcattgcagcagtgttgttttgtttgaagtgcacactctatggtacgaggtgtttagtatacccaagcagataggtgtcgatcgaacaggagcagggagaatacttccaacagttgaggtgttaccaaaccacttgagaattcatgagcactttaactctaaactctgaatttcaaagcttgatgtgaagtcctctagaatgtttacatttactaaggtgtgctgggtcctgtctcttttgactaatattttcgtaaacattaggctggagaaaggaaggaagcagtggtttccttagataactacagaattatactggtctctgggattactctctcagctgtattaaaatgaatttgtactttgaaaggaatgatattgacactaaaattttaaacatttaaattttttcataatctttcataaagaagtttaataataggtatattaactgaatttcattagttttttaaaataatattgtttgtgtatatatacatattaaaataaaaacatttacaacaaataaaatacttgaaattctaaaaaaaaaaaaaaaaaaa

SEQ ID NO: 4 - protein, 845 aa, Mus musculus
MGPRALSPLASLRLRWLLACGLLGPVLEAGRPDLEQTVHLSSYEIITPWRLTRERREALGPSSQQISYVIQAQGKQHIIHLERNTDLLPNDFVVYTYDKEGSLLSDHPNVQSHCHYRGYVEGVQNSAVAVSACFGLRGLLHLENASFGIEPLHNSSHFEHIFYPMDGIHQEPLRCGVSNRDTEKEGTQGDEEEHPSVTQLLRRRRAVLPQTRYVELFIVVDKERYDMMGRNQTAVREEMIRLANYLDSMYIMLNIRIVLVGLEIWTDRNPINIIGGAGDVLGNFVQWREKFLITRRRHDSAQLVLKKGFGGTAGMAFVGTVCSRSHAGGINVFGQITVETFASIVAHELGHNLGMNHDDGRECFCGAKSCIMNSGASGSRNFSSCSAEDFEKLTLNKGGSCLLNIPKPDEAYSAPSCGNKLVDPGEECDCGTAKECEVDPCCEGSTCKLKSFAECAYGDCCKDCQFLPGGSMCRGKTSECDVPEYCNGSSQFCPPDVFIQNGYPCQNSKAYCYNGMCQYYDAQCQVIFGSKAKAAPRDCFIEVNSKGDRFGNCGFSGSEYKKCATGNALCGKLQCENVQDMPVFGIVPAIIQTPSRGTKCWGVDFQLGSDVPDPGMVNEGTKCDAGKICRNFQCVNASVLNYDCDIQGKCHGHGVCNSNKNCHCEDGWAPPHCDTKGYGGSVDSGPTYNAKSTALRDGLLVFFFLIVPLVAAAIFLFIKRDELRKTFRKKRSQMSDGRNQANVSRQPGDPSISRPPGGPNVSRPPGGPGVSRPPGGPGVSRPPGGPGVSRPPPGHGNRFPVPTYAAKQPAQFPSRPPPPQPKISSQGNLIPARPAPAPPLYSSLT

SEQ ID NO: 5 - DNA, 2806 bp, Homo sapiens
  CDS (301)..(1839)
aggcggaggtctgagtttcgaaggagggggggagagaagagggaacgagcaagggaaggaaagcggggaaaggaggaaggaaacgaacgagggggagggaggtccctgttttggaggagctaggagcgttgccggcccctgaagtggagcgagagggaggtgcttcgccgtttctcctgccaggggaggtcccggcttcccgtggaggctccggaccaagccccttcagcttctccctccggatcgatgtgctgctgttaacccgtgaggaggcggcggcggcggcagcggcagcggaagatggtgttgctgagagtgttaattctgctcctctcctgggcggcggggatgggaggtcagtatgggaatcctttaaataaatatatcagacattatgaaggattatcttacaatgtggattcattacaccaaaaacaccagcgtgccaaaagagcagtctcacatgaagaccaatttttacgtctagatttccatgcccatggaagacatttcaacctacgaatgaagagggacacttcccttttcagtgatgaatttaaagtagaaacatcaaataaagtacttgattatgatacctctcatatttacactggacatatttatggtgaagaaggaagttttagccatgggtctgttattgatggaagatttgaaggattcatccagactcgtggtggcacattttatgttgagccagcagagagatatattaaagaccgaactctgccatttcactctgtcatttatcatgaagatgatattaactatccccataaatacggtcctcaggggggctgtgcagatcattcagtatttgaaagaatgaggaaataccagatgactggtgtagaggaagtaacacagatacctcaagaagaacatgctgctaatggtccagaacttctgaggaaaaaacgtacaacttcagctgaaaaaaatacttgtcagctttatattcagactgatcatttgttctttaaatattacggaacacgagaagctgtgattgcccagatatccagtcatgttaaagcgattgatacaatttaccagaccacagacttctccggaatccgtaacatcagtttcatggtgaaacgcataagaatcaatacaactgctgatgagaaggaccctacaaatcctttccgtttcccaaatattggtgtggagaagtttctggaattgaattctgagcagaatcatgatgactactgtttggcctatgtcttcacagaccgagattttgatgatggcgtacttggtctggcttgggttggagcaccttcaggaagctctggaggaatatgtgaaaaaagtaaactctattcagatggtaagaagaagtccttaaacactggaattattactgttcagaactatgggtctcatgtacctcccaaagtctctcacattacttttgctcacgaagttggacataactttggatccccacatgattctggaacagagtgcacaccaggagaatctaagaatttgggtcaaaaagaaaatggcaattacatcatgtatgcaagagcaacatctggggacaaacttaacaacaataaattctcactctgtagtattagaaatataagccaagttcttgagaagaagagaaacaactgttttgttgaatctggccaacctatttgtggaaatggaatggtagaacaaggtgaagaatgtgattgtggctatagtgaccagtgtaaagatgaatgctgcttcgatgcaaatcaaccagagggaagaaaatgcaaactgaaacctgggaaacagtgcagcacagtgtgcattcaagtcaaagtctgagaagtgtcgggatgattcagactgtgcaagggaaggaatatgtaatggcttcacagctctctgcccagcatctgaccctaaaccaaacttcacagactgtaataggcatacacaagtgtgcattaatgggcaatgtgcaggttctatctgtgagaaatatggcttagaggagtgtacgtgtgccagttctgatggcaaagatgataaagaattatgccatgtatgctgtatgaagaaaatggacccatcaacttgtgccagtacagggtctgtgcagtggagtaggcacttcagtggtcgaaccatcaccctgcaacctggatccccttgcaacgattttagaggttactgtgatgttttcatgcggtgcagattagtagatgctgatggtcctctagctaggcttaaaaaagcaatttttagtccagagctctatgaaaacattgctgaatggattgtggctcattggtgggcagtattacttatgggaattgctctgatcatgctaatggctggatttattaagatatgcagtgttcatactccaagtagtaatccaaagttgcctcctcctaaaccacttccaggcactttaaagaggaggagacctccacagcccattcagcaaccccagcgtcagcggccccgagagagttatcaaatgggacacatgagacgctaactgcagcttttgccttggttcttcctagtgcctacaatgggaaaacttcactccaaagagaaacctattaagtcatcatctccaaactaaaccctcacaagtaacagttgaagaaaaaatggcaagagatcatatcctcagaccaggtggaattacttaaattttaaagcctgaaaattccaatttgggggtgggaggtggaaaaggaacccaattttcttatgaacagatatttttaacttaatggcacaaagtcttagaatattattatgtgccccgtgttcc

SEQ ID NO: 6 - protein, 512 aa, Homo sapiens [sequence omitted]

SEQ ID NO: 7 - DNA, 2311 bp, Mus musculus
  CDS (35)..(2284)
gtacaaaaaagcagaagggccgtcaaggcccaccatggtgttgccgacagtgttaattctgctcctctcctgggcggcggggctgggaggtcagtatggaaatcctttaaataaatatattagacattatgaaggattatcttacaatgtggattcattacaccaaaaacaccagcgtgccaaacgagcagtctcacatgaggaccagtttttacttctagatttccatgctcatggaagacagttcaacctacgaatgaagagggacacttccctttttagtgatgaatttaaagtagaaacatcaaataaagtacttgattatgatacctctcatatttacactggacatatttatggtgaagaaggaagctttagtcatgggtctgtcattgatggaagatttgaaggtttcatcaagactcgtggtggcacgttttacattgagccagcagagagatacattaaagatcgaatcctgccatttcactctgtcatttatcatgaagatgatattaactatccccataaatacggcccacaggggggctgtgcagatcactccgtttttgaaaggatgaggaagtaccaaatgactggagtagaggaaggagcccgggcacatccagagaagcatgctgctagtagtggtcctgagctcctgaggaaaaaacgcacaactctggctgaaagaaatacttgtcagctctatatccagacagatcacctgttctttaaatactatggaacacgagaagctgtgattgctcagatatccagtcatgttaaagcaattgatacaatttaccagactacagacttctccggaatccgtaacatcagcttcatggtgaaacgcataagaatcaatacaacctctgatgaaaaagaccctacaaatcctttccgtttcccaaatattggtgtggagaagttcctggagttgaattctgagcagaatcatgatgactactgcctggcctatgtcttcacagaccgggattttgatgatggtgttcttggtctggcctgggttggagcaccttcaggaagctctgggggaatatgtgagaaaagcaagttgtattcagatggcaagaagaagtcattgaacacaggcatcattactgttcagaactatggctcccatgtgcctcccaaagtctctcatattacgtttgctcatgaagttggacataactttggatctccacatgattctggaacagagtgtactccaggagagtctaagaacttaggacaaaaagaaaatggcaattacatcatgtatgcaagagcaacatctggggacaaacttaacaacaacaaattttcactctgcagcattagaaacataagccaagtgcttgagaagaagaggaacaactgttttgttgaatctggccagcctatctgtggaaacgggatggtggaacaaggggaagagtgtgactgtggctacagtgaccagtgcaaagatgattgctgcttcgatgccaaccagccagaggggaagaaatgcaagctgaagcctgggaagcagtgcagtccgagtcaaggaccctgctgtacagcacagtgtgcattcaagtcaaagtctgaaaagtgccgggatgattctgactgtgcaaaggaagggatatgcaatggcttcacagccctttgcccagcatctgatcccaagcccaactttacagactgtaacaggcacacacaagtgtgcattaatgggcaatgtgcaggttctatttgtgaaaagtatgacttggaggagtgcacctgtgccagctctgatggcaaagatgataaggaattatgccatgtttgctgcatgaagaaaatggctccatcaacttgtgccagtacaggctctttgcagtggagcaagcagttcagtggtcggactatcactctgcagccgggctctccatgtaatgacttcagaggctactgtgatgttttcatgcggtgcagattagtagatgctgatggccctctagctaggctgaaaaaagccatttttagtccacaactctatgaaaacattgctgagtggattgtggctcactggtgggcagtactgcttatgggaattgccctgatcatgttaatggctggatttatcaagatttgcagtgttcacactccaagtagtaatccaaagttgccgcctcctaaaccacttccaggcactttaaagaggaggagaccgccacagcccattcagcagcccccgcgtcagaggccccgagagagttatcaaatgggacacatgcgacgctagggcctcatgggcccagctttcttgtac

SEQ ID NO: 8 - protein, 749 aa, Mus musculus
MVLPTVLILLLSWAAGLGGQYGNPLNKYIRHYEGLSYNVDSLHQKHQRAKRAVSHEDQFLLLDFHAHGRQFNLRMKRDTSLFSDEFKVETSNKVLDYDTSHIYTGHIYGEEGSFSHGSVIDGRFEGFIKTRGGTFYIEPAERYIKDRILPFHSVIYHEDDINYPHKYGPQGGCADHSVFERMRKYQMTGVEEGARAHPEKHAASSGPELLRKKRTTLAERNTCQLYIQTDHLFFKYYGTREAVIAQISSHVKAIDTIYQTTDFSGIRNISFMVKRIRINTTSDEKDPTNPFRFPNIGVEKFLELNSEQNHDDYCLAYVFTDRDFDDGVLGLAWVGAPSGSSGGICEKSKLYSDGKKKSLNTGIITVQNYGSHVPPKVSHITFAHEVGHNFGSPHDSGTECTPGESKNLGQKENGNYIMYARATSGDKLNNNKFSLCSIRNISQVLEKKRNNCFVESGQPICGNGMVEQGEECDCGYSDQCKDDCCFDANQPEGKKCKLKPGKQCSPSQGPCCTAQCAFKSKSEKCRDDSDCAKEGICNGFTALCPASDPKPNFTDCNRHTQVCINGQCAGSICEKYDLEECTCASSDGKDDKELCHVCCMKKMAPSTCASTGSLQWSKQFSGRTITLQPGSPCNDFRGYCDVFMRCRLVDADGPLARLKKAIFSPQLYENIAEWIVAHWWAVLLMGIALIMLMAGFIKICSVHTPSSNPKLPPPKPLPGTLKRRRPPQPIQQPPRQRPRESYQMGHMRR

SEQ ID NO: 9 - DNA, 3389 bp, Homo sapiens
  CDS (78)..(2552)
ccgatgtgagcagttttccgaaaccccgtcaggcgaaggctgcccagagaggtggagtcggtagcggggccgggaacatgaggcagtctctcctattcctgaccagcgtggttcctttcgtgctggcgccgcgacctccggatgacccgggcttcggcccccaccagagactcgagaagcttgattctttgctctcagactacgatattctctctttatctaatatccagcagcattcggtaagaaaaagagatctacagacttcaacacatgtagaaacactactaactttttcagctttgaaaaggcattttaaattatacctgacatcaagtactgaacgtttttcacaaaatttcaaggtcgtggtggtggatggtaaaaacgaaagcgagtacactgtaaaatggcaggacttcttcactggacacgtggttggtgagcctgactctagggttctagcccacataagagatgatgatgttataatcagaatcaacacagatggggccgaatataacatagagccactttggagatttgttaatgataccaaagacaaaagaatgttagtttataaatctgaagatatcaagaatgtttcacgtttgcagtctccaaaagtgtgtggttatttaaaagtggataatgaagagttgctcccaaaagggttagtagacagagaaccacctgaagagcttgttcatcgagtgaaaagaagagctgacccagatcccatgaagaacacgtgtaaattattggtggtagcagatcatcgcttctacagatacatgggcagaggggaagagagtacaactacaaattacttaatagagctaattgacagagttgatgacatctatcggaacacttcatgggataatgcaggttttaaaggctatggaatacagatagagcagattcgcattctcaagtctccacaagaggtaaaacctggtgaaaagcactacaacatggcaaaaagttacccaaatgaagaaaaggatgcttgggatgtgaagatgttgctagagcaatttagctttgatatagctgaggaagcatctaaagtttgcttggcacaccttttcacataccaagattttgatatgggaactcttggattagcttatgttggctctcccagagcaaacagccatggaggtgtttgtccaaaggcttattatagcccagttgggaagaaaaatatctatttgaatagtggtttgacgagcacaaagaattatggtaaaaccatccttacaaaggaagctgacctggttacaactcatgaattgggacataattttggagcagaacatgatccggatggtctagcagaatgtgccccgaatgaggaccagggagggaaatatgtcatgtatcccatagctgtgagtggcgatcacgagaacaataagatgttttcaaactgcagtaaacaatcaatctataagaccattgaaagtaaggcccaggagtgttttcaagaacgcagcaataaagtttgtgggaactcgagggtggatgaaggagaagagtgtgatcctggcatcatgtatctgaacaacgacacctgctgcaacagcgactgcacgttgaaggaaggtgtccagtgcagtgacaggaacagtccttgctgtaaaaactgtcagtttgagactgcccagaagaagtgccaggaggcgattaatgctacttgcaaaggcgtgtcctactgcacaggtaatagcagtgagtgcccgcctccaggaaatgctgaagatgacactgtttgcttggatcttggcaagtgtaaggatgggaaatgcatccctttctgcgagagggaacagcagctggagtcctgtgcatgtaatgaaactgacaactcctgcaaggtgtgctgcagggacctttccggccgctgtgtgccctatgtcgatgctgaacaaaagaacttatttttgaggaaaggaaagccctgtacagtaggattttgtgacatgaatggcaaatgtgagaaacgagtacaggatgtaattgaacgattttgggatttcattgaccagctgagcatcaatacttttggaaagtttttagcagacaacatcgttgggtctgtcctggttttctccttgatattttggattcctttcagcattcttgtccattgtgtggataagaaattggataaacagtatgaatctctgtctctgtttcaccccagtaacgtcgaaatgctgagcagcatggattctgcatcggttcgcattatcaaaccctttcctgcgccccagactccaggccgcctgcagcctgcccctgtgatcccttcggcgccagcagctccaaaactggaccaccagagaatggacaccatccaggaagaccccagcacagactcacatatggacgaggatgggtttgagaaggaccccttcccaaatagcagcacagctgccaagtcatttgaggatctcacggaccatccggtcaccagaagtgaaaaggctgcctcctttaaactgcagcgtcagaatcgtgttgacagcaaagaaacagagtgctaatttagttctcagctcttctgacttaagtgtgcaaaatatttttatagatttgacctacaatcaatcacagcttatattttgtgaagactgggaagtgacttagcagatgctggtcatgtgtttgaacttcctgcaggtaaacagttcttgtgtggtttggcccttctccttttgaaaaggtaaggtgaaggtgaatctagcttattttgaggctttcaggttttagtttttaaaatatcttttgacctgtggtgcaaaagcagaaaatacagctggattgggttatgagtatttacgtttttgtaaattaatcttttatattgataacagcactgactagggaaatgatcagttttttttttatacactgtaatgaaccgctgaatatgaggcatttggcatttatttgtgatgacaactggaatagtttttttttttttttttgccttcaactaaaaacaaaggagataaatctagtatacattgtctctaaattgtgggtctatttctagttattacccagagtttttatgtagcagggaaaatatatatctaaatttagaaatcgtttgggttaatatggctcttcataattctaagactaatgctctctagaaacctaaccacctaccttacagtgagggctatacatggtagccagttgaatttatggaatctaccaactgtttagggccctgatttgctgggcagtttttctgtattttataagtatcttcatgtatccctgttactgatagggatacatgctcttagaaaattcactattggctgggagtggtggctcatgcctgtaatcccagcacttggagaggctga

SEQ ID NO: 10 - protein, 824 aa, Homo sapiens
MRQSLLFLTSVVPFVLAPRPPDDPGFGPHQRLEKLDSLLSDYDILSLSNIQQHSVRKRDLQTSTHVETLLTFSALKRHFKLYLTSSTERFSQNFKVVVVDGKNESEYTVKWQDFFTGHVVGEPDSRVLAHIRDDDVIIRINTDGAEYNIEPLWRFVNDTKDKRMLVYKSEDIKNVSRLQSPKVCGYLKVDNEELLPKGLVDREPPEELVHRVKRRADPDPMKNTCKLLVVADHRFYRYMGRGEESTTTNYLIELIDRVDDIYRNTSWDNAGFKGYGIQIEQIRILKSPQEVKPGEKHYNMAKSYPNEEKDAWDVKMLLEQFSFDIAEEASKVCLAHLFTYQDFDMGTLGLAYVGSPRANSHGGVCPKAYYSPVGKKNIYLNSGLTSTKNYGKTILTKEADLVTTHELGHNFGAEHDPDGLAECAPNEDQGGKYVMYPIAVSGDHENNKMFSNCSKQSIYKTIESKAQECFQERSNKVCGNSRVDEGEECDPGIMYLNNDTCCNSDCTLKEGVQCSDRNSPCCKNCQFETAQKKCQEAINATCKGVSYCTGNSSECPPPGNAEDDTVCLDLGKCKDGKCIPFCEREQQLESCACNETDNSCKVCCRDLSGRCVPYVDAEQKNLFLRKGKPCTVGFCDMNGKCEKRVQDVIERFWDFIDQLSINTFGKFLADNIVGSVLVFSLIFWIPFSILVHCVDKKLDKQYESLSLFHPSNVEMLSSMDSASVRIIKPFPAPQTPGRLQPAPVIPSAPAAPKLDHQRMDTIQEDPSTDSHMDEDGFEKDPFPNSSTAAKSFEDLTDHPVTRSEKAASFKLQRQNRVDSKETEC

SEQ ID NO: 11 - DNA, 3648 bp, Mus musculus
  CDS (80)..(2563)
atcacctccgctcccaatgtgagcagtttcccgaacgctctttcggagaaggttgcccagagaggtggtggacgggaacatgaggcggcgtctcctcatcctgaccactttggtgcctttcgtcctggcaccccgacctccggaggaagcaggctctggcgcccatccgcgacttgagaagcttgattctttgctctcagactacgacatcctctccttagctaatattcagcagcactccataaggaaaagggatctacagtctgcgacacacttagaaacattactaactttttcagctttgaaaaggcattttaaattatacttgacatcaagtaccgaacgcttttcacaaaacttgagagtcgtggtggtggacgggaaagaagaaagcgagtacagcgtgaagtggcaggacttcttcagtggtcacgtggttggtgagcctgactctagggttctagcccacataggagatgatgatgttacagtgagaatcaacacagatggggcagaatataacgtagagccactttggaggtttgtcaatgatactaaagataaacgaatgctggtgtataagtctgaagatatcaaggatttttcacgtttgcagtctccaaaagtatgtggttatttaaatgcagatagtgaagagctgcttccaaaagggctcatagacagagagccatctgaagagtttgttcgtcgagtgaagagacgagctgaacctaaccccttgaagaatacttgtaaattactggtggtagcagatcatcgattttataaatacatgggccgtggagaagagagcaccactacaaattacttaatagagctaattgaccgagttgatgacatataccggaacacgtcgtgggataatgcagggtttaaagggtatggagtgcagatagagcagattcgaattctcaagtctccacaagaggtaaaacctggtgaaagacacttcaatatggcaaaaagtttcccaaacgaagagaaggatgcttgggatgtgaagatgctattagagcaatttagctttgatatagctgaagaagcatccaaagtctgcctggctcatcttttcacgtaccaggattttgatatgggaactcttggattagcttacgttggttctcccagagcaaacagtcatggaggggtttgtccaaaagcttattacaacccaactgtgaagaaaaacatctatttaaatagtggtctgactagtactaaaaattatggtaaaactattctcacaaaggaagctgacctggttacaactcatgaattgggacacaattttggagcagaacatgaccctgatgggctagcagaatgtgctccaaatgaggaccaaggaggaaagtatgtcatgtatcccatagctgtgagcggtgaccatgagaataataagatgttttcaaactgcagtaaacagtccatctacaagaccatagaaagtaaggctcaagagtgcttccaggagcgcagcaacaaggtgtgtggcaactccagggtggatgaaggagaggagtgtgacccgggtattatgtacctgaacaacgacacctgctgcaatagtgactgcacactgaagccgggtgtgcagtgcagtgataggaacagtccttgctgtaaaaactgtcagtttgagacggcgcagaagaagtgccaggaggctattaatgctacatgcaaaggagtgtcttactgcacagggaatagcagtgagtgccccccacccggagatgctgaagatgacactgtgtgcttggaccttggcaagtgcaaggctgggaagtgcatccctttctgcaagagggagcaggagctggagtcctgcgcatgcgttgacactgacaactcgtgcaaggtgtgctgcaggaacctttctggcccgtgtgtgccgtacgtcgatgcagagcaaaagaacttgtttttgaggaaagggaagccatgtacagtagggttttgcgacatgaatggcaaatgtgagaaacgagtacaggacgtaattgagcgattttgggatttcattgaccagctgagcatcaacacttttgggaagtttctggcagataacatcgttgggtctgttctggttttctccttgatattttggattcctttcagcattcttgtccactgtgtggataagaaactggacaagcagtatgaatccctgtctctgtttcatcacagtaacattgagatgctgagcagcatggactcagcatctgttcgcatcatcaagccctttcctgcaccccagactccaggtcgtctgcaggccctgcagccagctgccatgatgccgccagtacctgcggctccaaaactggaccaccagcggatggacaccatccaggaagaccccagcacagactcacatgcagatgatgacggatttgagaaggaccccttccccaacagcagcacagctgccaagtcctttgaggacctcacagaccacccagtcaccaggagcgaaaaggcggcctcattcaagctgcagcgtcagagccgagttgacagcaaagagacagagtgctagtggggaaccttggcctgctctaggacatatacctgcagatgttccatagagctgacctgaatcaaaacatagattataatgatctgagaaacggggaagcaacttagcagatgctggtcatgtgctatgaccttcacatgacctcctatgtatgtaggccctttgaagaggtgaggtaaatctggcttatgtaaggctttcaggttttgggtttttcttttctaatctaaaatctcctttgccctgtggtgcagaagcagaaagtaaggctggaccccgttcctggtgacagtgctgttaagtcttcagtctgtttttctgtatcctctgactacagtgaagcacttaacagtgaggataactggaacacagacatacttgtttgtttgttttgtttttgtttttgttttttttttgaccctcaactaaaagaggagcaagagaaacctgcttgtatgttgtctccaaatgcggcctgtcttggcactgaattctttgtagatggagagacctgcctacacttagggccatctacattaagagcagtcctttccaagaggaacagtttatagaaacagccacatgaacttcgagatggcctattaactgttcagccccagattcactgggcagtttttccattttaccaatgtgtcgtctttaaactgtttgccttaaaaaaaaaaatcaatattgggctggagagttggccccacagttaagagcagttggtctcattctcagcacccacatggtggctctcaactctgtaactccaatcccagaggagatgacatcttctgaactctaggacacgaggcatacattggtgcaggcaaaacaaagccaagtgctgtaaaatgtggcaataaccacaaaacctatagtgcctcacagtatggactgagtggtgaatagacactcttcccggaaccacttctggacaggccgggatgtgctggctggctaggaagagggggtgggagtgggagagtggtgctgccattggggcccagccctagaagaactgaccttcagtctcggcaagtagacacccacgtgacctctggctctggtgcttcagg

SEQ ID NO: 12 - protein, 827 aa, Mus musculus
MRRRLLILTTLVPFVLAPRPPEEAGSGAHPRLEKLDSLLSDYDILSLANIQQHSIRKRDLQSATHLETLLTFSALKRHFKLYLTSSTERFSQNLRVVVVDGKEESEYSVKWQDFFSGHVVGEPDSRVLAHIGDDDVTVRINTDGAEYNVEPLWRFVNDTKDKRMLVYKSEDIKDFSRLQSPKVCGYLNADSEELLPKGLIDREPSEEFVRRVKRRAEPNPLKNTCKLLVVADHRFYKYMGRGEESTTTNYLIELIDRVDDIYRNTSWDNAGFKGYGVQIEQIRILKSPQEVKPGERHFNMAKSFPNEEKDAWDVKMLLEQFSFDIAEEASKVCLAHLFTYQDFDMGTLGLAYVGSPRANSHGGVCPKAYYNPTVKKNIYLNSGLTSTKNYGKTILTKEADLVTTHELGHNFGAEHDPDGLAECAPNEDQGGKYVMYPIAVSGDHENNKMFSNCSKQSIYKTIESKAQECFQERSNKVCGNSRVDEGEECDPGIMYLNNDTCCNSDCTLKPGVQCSDRNSPCCKNCQFETAQKKCQEAINATCKGVSYCTGNSSECPPPGDAEDDTVCLDLGKCKAGKCIPFCKREQELESCACVDTDNSCKVCCRNLSGPCVPYVDAEQKNLFLRKGKPCTVGFCDMNGKCEKRVQDVIERFWDFIDQLSINTFGKFLADNIVGSVLVFSLIFWIPFSILVHCVDKKLDKQYESLSLFHHSNIEMLSSMDSASVRIIKPFPAPQTPGRLQALQPAAMMPPVPAAPKLDHQRMDTIQEDPSTDSHADDDGFEKDPFPNSSTAAKSFEDLTDHPVTRSEKAASFKLQRQSRVDSKETEC

SEQ ID NO: 13 - DNA, 2802 bp, Homo sapiens
  CDS (266)..(1657)
ggttctcgggcggggcctgggacaggcagctccggggtccgcggtttcacatcggaaacaaaacagcggctggtctggaaggaacctgagctacgagccgcggcggcagcggggcggcggggaagcgtatacctaatctgggagcctgcaagtgacaacagcctttgcggtccttagacagcttggcctggaggagaacacatgaaagaaagaacctcaagaggctttgttttctgtgaaacagtatttctatacagttgctccaatgacagagttacctgcaccgttgtcctacttccagaatgcacagatgtctgaggacaaccacctgagcaatactaatgacaatagagaacggcaggagcacaacgacagacggagccttggccaccctgagccattatctaatggacgaccccagggtaactcccggcaggtggtggagcaagatgaggaagaagatgaggagctgacattgaaatatggcgccaagcatgtgatcatgctctttgtccctgtgactctctgcatggtggtggtcgtggctaccattaagtcagtcagcttttatacccggaaggatgggcagctaatctataccccattcacagaagataccgagactgtgggccagagagccctgcactcaattctgaatgctgccatcatgatcagtgtcattgttgtcatgactatcctcctggtggttctgtataaatacaggtgctataaggtcatccatgcctggcttattatatcatctctattgttgctgttctttttttcattcatttacttgggggaagtgtttaaaacctataacgttgctgtggactacattactgttgcactcctgatctggaattttggtgtggtgggaatgatttccattcactggaaaggtccacttcgactccagcaggcatatctcattatgattagtgccctcatggccctggtgtttatcaagtacctccctgaatggactgcgtggctcatcttggctgtgatttcagtatatgatttagtggctgttttgtgtccgaaaggtccacttcgtatgctggttgaaacagctcaggagagaaatgaaacgctttttccagctctcatttactcctcaacaatggtgtggttggtgaatatggcagaaggagacccggaagctcaaaggagagtatccaaaaattccaagtataatgcagaaagcacagaaagggagtcacaagacactgttgcagagaatgatgatggcgggttcagtgaggaatgggaagcccagagggacagtcatctagggcctcatcgctctacacctgagtcacgagctgctgtccaggaactttccagcagtatcctcgctggtgaagacccagaggaaaggggagtaaaacttggattgggagatttcattttctacagtgttctggttggtaaagcctcagcaacagccagtggagactggaacacaaccatagcctgtttcgtagccatattaattggtttgtgccttacattattactccttgccattttcaagaaagcattgccagctcttccaatctccatcacctttgggcttgttttctactttgccacagattatcttgtacagccttttatggaccaattagcattccatcaattttatatctagcatatttgcggttagaatcccatggatgtttcttctttgactataacaaaatctggggaggacaaaggtgattttcctgtgtccacatctaacaaagtcaagattcccggctggacttttgcagcttccttccaagtcttcctgaccaccttgcactattggactttggaaggaggtgcctatagaaaacgattttgaacatacttcatcgcagtggactgtgtccctcggtgcagaaactaccagatttgagggacgaggtcaaggagatatgataggcccggaagttgctgtgccccatcagcagcttgacgcgtggtcacaggacgatttcactgacactgcgaactctcaggactaccgttaccaagaggttaggtgaagtggtttaaaccaaacggaactcttcatcttaaactacacgttgaaaatcaacccaataattctgtattaactgaattctgaacttttcaggaggtactgtgaggaagagcaggcaccagcagcagaatggggaatggagaggtgggcaggggttccagcttccctttgattttttgctgcagactcatcctttttaaatgagacttgttttcccctctctttgagtcaagtcaaatatgtagattgcctttggcaattcttcttctcaagcactgacactcattaccgtctgtgattgccatttcttcccaaggccagtctgaacctgaggttgctttatcctaaaagttttaacctcaggttccaaattcagtaaattttggaaacagtacagctatttctcatcaattctctatcatgttgaagtcaaatttggattttccaccaaattctgaatttgtagacatacttgtacgctcacttgccccagatgcctcctctgtcctcattcttctctcccacacaagcagtctttttctacagccagtaaggcagctctgtcgtggtagcagatggtcccattattctagggtcttactctttgtatgatgaaaagaatgtgttatgaatcggtgctgtcagccctgctgtcagaccttcttccacagcaaatgagatgtatgcccaaagacggtagaattaaagaagagtaaaatggctgttgaagcaaaaaaaaaaaaaaaaaaaaaaaaaaaaaaaaa

SEQ ID NO: 14 - protein, 463 aa, Homo sapiens
MTELPAPLSYFQNAQMSEDNHLSNTNDNRERQEHNDRRSLGHPEPLSNGRPQGNSRQVVEQDEEEDEELTLKYGAKHVIMLFVPVTLCMVVVVATIKSVSFYTRKDGQLIYTPFTEDTETVGQRALHSILNAAIMISVIVVMTILLVVLYKYRCYKVIHAWLIISSLLLLFFFSFIYLGEVFKTYNVAVDYITVALLIWNFGVVGMISIHWKGPLRLQQAYLIMISALMALVFIKYLPEWTAWLILAVISVYDLVAVLCPKGPLRMLVETAQERNETLFPALIYSSTMVWLVNMAEGDPEAQRRVSKNSKYNAESTERESQDTVAENDDGGFSEEWEAQRDSHLGPHRSTPESRAAVQELSSSILAGEDPEERGVKLGLGDFIFYSVLVGKASATASGDWNTTIACFVAILIGLCLTLLLLAIFKKALPALPISITFGLVFYFATDYLVQPFMDQLAFHQFYI

SEQ ID NO: 15 - DNA, 2750 bp, Mus musculus
  CDS (231)..(1634)
cccacgcgtccgcggacgcgtgggtcgcgagtattcgtcggaaacaaaacagcggcagctgaggcggaaacctaggctgcgagccggccgcccgggcgcggagagagaaggaaccaacacaagacagcagcccttcgaggtctttaggcagcttggaggagaacacatgagagaaagaatcccaagaggttttgttttctttgagaaggtatttctgtccagctgctccaatgacagagatacctgcacctttgtcctacttccagaatgcccagatgtctgaggacagccactccagcagcgccatccggagccagaatgacagccaagaacggcagcagcagcatgacaggcagagacttgacaaccctgagccaatatctaatgggcggccccagagtaactcaagacaggtggtggaacaagatgaggaggaagacgaagagctgacattgaaatatggagccaagcatgtcatcatgctctttgtccccgtgaccctctgcatggtcgtcgtcgtggccaccatcaaatcagtcagcttctatacccggaaggacggtcagctaatctacaccccattcacagaagacactgagactgtaggccaaagagccctgcactcgatcctgaatgcggccatcatgatcagtgtcattgtcattatgaccatcctcctggtggtcctgtataaatacaggtgctacaaggtcatccacgcctggcttattatttcatctctgttgttgctgttctttttttcgttcatttacttaggggaagtatttaagacctacaatgtcgccgtggactacgttacagtagcactcctaatctggaattttggtgtggtcgggatgattgccatccactggaaaggcccccttcgactgcagcaggcgtatctcattatgatcagtgccctcatggccctggtatttatcaagtacctccccgaatggaccgcatggctcatcttggctgtgatttcagtatatgatttggtggctgttttatgtcccaaaggcccacttcgtatgctggttgaaacagctcaggaaagaaatgagactctctttccagctcttatctattcctcaacaatggtgtggttggtgaatatggctgaaggagacccagaagcccaaaggagggtacccaagaaccccaagtataacacacaaagagcggagagagagacacaggacagtggttctgggaacgatgatggtggcttcagtgaggagtgggaggcccaaagagacagtcacctggggcctcatcgctccactcccgagtcaagagctgctgtccaggaactttctgggagcattctaacgagtgaagacccggaggaaagaggagtaaaacttggactgggagatttcattttctacagtgttctggttggtaaggcctcagcaaccgccagtggagactggaacacaaccatagcctgctttgtagccatactgatcggcctgtgccttacattactcctgctcgccattttcaagaaagcgttgccagccctccccatctccatcaccttcgggctcgtgttctacttcgccacggattaccttgtgcagcccttcatggaccaacttgcattccatcagttttatatctagcctttctgcagttagaacatggatgtttcttctttgattatcaaaaacacaaaaacagagagcaagcccgaggaggagactggtgactttcctgtgtcctcagctaacaaaggcaggactccagctggacttctgcagcttccttccgagtctccctagccacccgcactactggactgtggaaggaagcgtctacagagaacggtttccaacatccatcgctgcagcagacggtgtccctcagtgacttgagagacaaggacaaggaaatgtgctgggccaaggagctgccgtgctctgctagctttgacccgtgggcatggagatttacccgcactgtgaactctctaaaggttaaacaaagtgaggtgaaccaaacagagctgccatcttccacaccatgttggaaataaaacacgtcctagctgaacccttactgtccaggaggttccgtgtggaggtggcactgggccgggcctccctctcaggctcttttgctgcccacttgtaagtttaaataaggacaccgccctacacaaacctcacccctgtcacatcagtgactctgaccacttttgttctcaaactctctcactattatctgtggttgccgtttcttcccaaggccagcctggacgaatttggggttgctctatcctgagagttgtaacctcaacttccaaagtttatattttcttgaaatgatggatctattgctcaacagtccctgtcatccttaagtgacttctgggtttcccacaaattcctcacttttagacacactctaagcttacttctggcctggatgcttcctctccctgtctctcccttgccccacagcggttccctgacagcagacaaggcagctctgggaggtagctagtatccaataacccaggggtttcctcatgtgatgcaaatactacgtgtccaaccaatcagtgctgtcaacgggctgccatagctccttcgatggcaaataggatgtgtgcccaaagaattaaagcgatgagtggctggtgaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 16 - protein, 467 aa, Mus musculus
MTEIPAPLSYFQNAQMSEDSHSSSAIRSQNDSQERQQQHDRQRLDNPEPISNGRPQSNSRQVVEQDEEEDEELTLKYGAKHVIMLFVPVTLCMVVVVATIKSVSFYTRKDGQLIYTPFTEDTETVGQRALHSILNAAIMISVIVIMTILLVVLYKYRCYKVIHAWLIISSLLLLFFFSFIYLGEVFKTYNVAVDYVTVALLIWNFGVVGMIAIHWKGPLRLQQAYLIMISALMALVFIKYLPEWTAWLILAVISVYDLVAVLCPKGPLRMLVETAQERNETLFPALIYSSTMVWLVNMAEGDPEAQRRVPKNPKYNTQRAERETQDSGSGNDDGGFSEEWEAQRDSHLGPHRSTPESRAAVQELSGSILTSEDPEERGVKLGLGDFIFYSVLVGKASATASGDWNTTIACFVAILIGLCLTLLLLAIFKKALPALPISITFGLVFYFATDYLVQPFMDQLAFHQFYI

SEQ ID NO: 17 - DNA, 21 bp, Artificial Sequence
  Synthetic - PS1 siRNA 1-1
aaggcccacttcgtatgctgg

SEQ ID NO: 18 - DNA, 19 bp, Artificial Sequence
  Synthetic - PS1 siRNA 4-11
ggaccaacttgcattccat

SEQ ID NO: 19 - DNA, 1864 bp, Homo sapiens
  CDS (92)..(1648)
gccccgcgcgccggccgagtcgctgagccgcggctgccggacgggacgggaccggctaggctgggcgcgccccccgggccccgccgtgggcatgggcgcactggcccgggcgctgctgctgcctctgctggcccagtggctcctgcgcgccgccccggagctggcccccgcgcccttcacgctgcccctccgggtggccgcggccacgaaccgcgtagttgcgcccaccccgggacccgggacccctgccgagcgccacgccgacggcttggcgctcgccctggagcctgccctggcgtcccccgcgggcgccgccaacttcttggccatggtagacaacctgcagggggactctggccgcggctactacctggagatgctgatcgggacccccccgcagaagctacagattctcgttgacactggaagcagtaactttgccgtggcaggaaccccgcactcctacatagacacgtactttgacacagagaggtctagcacataccgctccaagggctttgacgtcacagtgaagtacacacaaggaagctggacgggcttcgttggggaagacctcgtcaccatccccaaaggcttcaatacttcttttcttgtcaacattgccactatttttgaatcagagaatttctttttgcctgggattaaatggaatggaatacttggcctagcttatgccacacttgccaagccatcaagttctctggagaccttcttcgactccctggtgacacaagcaaacatccccaacgttttctccatgcagatgtgtggagccggcttgcccgttgctggatctgggaccaacggaggtagtcttgtcttgggtggaattgaaccaagtttgtataaaggagacatctggtatacccctattaaggaagagtggtactaccagatagaaattctgaaattggaaattggaggccaaagccttaatctggactgcagagagtataacgcagacaaggccatcgtggacagtggcaccacgctgctgcgcctgccccagaaggtgtttgatgcggtggtggaagctgtggcccgcgcatctctgattccagaattctctgatggtttctggactgggtcccagctggcgtgctggacgaattcggaaacaccttggtcttacttccctaaaatctccatctacctgagagatgagaactccagcaggtcattccgtatcacaatcctgcctcagctttacattcagcccatgatgggggccggcctgaattatgaatgttaccgattcggcatttccccatccacaaatgcgctggtgatcggtgccacggtgatggagggcttctacgtcatcttcgacagagcccagaagagggtgggcttcgcagcgagcccctgtgcagaaattgcaggtgctgcagtgtctgaaatttccgggcctttctcaacagaggatgtagccagcaactgtgtccccgctcagtctttgagcgagcccattttgtggattgtgtcctatgcgctcatgagcgtctgtggagccatcctccttgtcttaatcgtcctgctgctgctgccgttccggtgtcagcgtcgcccccgtgaccctgaggtcgtcaatgatgagtcctctctggtcagacatcgctggaaatgaatagccaggcctgacctcaagcaaccatgaactcagctattaagaaaatcacatttccagggcagcagccgggatcgatggtggcgctttctcctgtgcccacccgtcttcaatctctgttctgctcccagatgccttctagattcactgtcttttgattcttgattttcaagctttcaaatcctccctacttccaagaaaaaaaaaaaaaaaaaa

SEQ ID NO: 20 - protein, 518 aa, Homo sapiens
MGALARALLLPLLAQWLLRAAPELAPAPFTLPLRVAAATNRVVAPTPGPGTPAERHADGLALALEPALASPAGAANFLAMVDNLQGDSGRGYYLEMLIGTPPQKLQILVDTGSSNFAVAGTPHSYIDTYFDTERSSTYRSKGFDVTVKYTQGSWTGFVGEDLVTIPKGFNTSFLVNIATIFESENFFLPGIKWNGILGLAYATLAKPSSSLETFFDSLVTQANIPNVFSMQMCGAGLPVAGSGTNGGSLVLGGIEPSLYKGDIWYTPIKEEWYYQIEILKLEIGGQSLNLDCREYNADKAIVDSGTTLLRLPQKVFDAVVEAVARASLIPEFSDGFWTGSQLACWTNSETPWSYFPKISIYLRDENSSRSFRITILPQLYIQPMMGAGLNYECYRFGISPSTNALVIGATVMEGFYVIFDRAQKRVGFAASPCAEIAGAAVSEISGPFSTEDVASNCVPAQSLSEPILWIVSYALMSVCGAILLVLIVLLLLPFRCQRRPRDPEVVNDESSLVRHRWK

SEQ ID NO: 21 - DNA, 2351 bp, Mus musculus
  CDS (41)..(1585)
agaaccgcgctcgcagactcgccgagcccccgctgcggggatgggcgcgctgcttcgagcactcttgctcctggtgctggcgcagtggctcttgagtgcggtccccgcgctggcccccgcgcccttcacgctgcccctccaagtggccggggccacgaaccacagagcctcggctgttcccggactcgggacccccgagttgccccgggccgatggtctggccctcgcactggagcctgtcagggctactgccaacttcttggctatggtggacaaccttcagggggactctggccgcggctactacctagagatgctgatcgggacccctccgcagaaggtacagattcttgtggacactggaagcagtaacttcgctgtggcaggtgccccacactcctacatagacacctactttgactcagagagctccagcacataccactccaagggctttgatgtcactgtgaagtacacacagggaagctggactggctttgttggtgaggaccttgtcaccatcccaaaaggcttcaacagctctttcttggtcaatattgccactattttcgagtctgagaatttctttttgcctggtattaaatggaatggaatccttggacttgcttatgctgctttggccaagccatcaagctctctggagacattttttgattccctggtggcccaagcaaagattccagacattttctccatgcagatgtgcggggctggattgccagtagctggttctggtaccaacggaggtagtcttgtcctgggtgggattgaaccaagtttgtataaaggagatatctggtataccccaattaaagaggaatggtactatcaaatagaaatcctgaagttggaaattggaggccagaacctcaacctggactgcagagagtataacgcagacaaggccattgtggacagtggcaccacgctcctgcgcctgccccagaaggtgtttgatgcagtggtggaagctgtggcacgaacatctctgattccagagttttctgatggcttctggacaggggcccagctggcatgctggacaaattctgaaacgccatgggcatatttccctaagatttctatctacctgagagatgagaatgccagtcgctccttccggatcaccattctcccacagctctacattcagcccatgatgggagctggtttcaattatgaatgctaccgttttggtatctcctcttccacaaatgcgctggtgattggtgcgaccgtgatggaaggcttctacgtggtctttgacagagctcagaggagggtgggctttgcagtgagtccctgtgcagagattgaaggtaccacagtgtctgaaatttctgggcccttttccacggaagacatagccagcaactgtgttccagcacaggctctgaatgagcccatcttgtggattgtgtcctatgccctgatgagtgtgtgtggagccattctcctggttctgatcctcctcctgctgctcccgctgcactgccgtcatgccccccgagaccctgaggtagttaacgatgagtcctcactagtcagacatcgctggaaatgaagagcctcacctgaactccagcagccttgaactcagctcttccaagagggacacctccagttggcttctctgcctattagtcgggaacctcaactgtgcaactgaatgccttccagactgtatcttgattactcttgatttccaagctttcagatcttttctacttcagagagaaatgataataaaaacacctcattgtgaaccaaaacagtgcattgggcttcagactttatgggactggttatgccaaactgcccagtgtaccaccaaaaccaaaaacaacccaaacaaagaaaaaataggcaagccatggttagttactatattctcttcaacctcaggaaaaaatgaatgcattcagttgcttgctctcttgcctagaggaaacttttgatattgaccatattaaagggtattttgtgatagactactgcctagtctagaacaagtgagaaggcgttcaatgtcctagcaggagaagtagacttgagccagatgagagaatcacttagccttcttctgcacctgactgtactgcaggaccttgggtaggtctcaggctccccaacaatgagaatccacacacagatcacactagcccttgccaggaagttctttgcccatctaggaagagtgagatggcattctaatgaagtgtagtaactttggcacccaggttaagatgccactgcatgacttggccagtgttctgagttagggtagggttcaggcagcttgattcccactattggcctggcctctgtga

SEQ ID NO: 22 - protein, 514 aa, Mus musculus
MGALLRALLLLVLAQWLLSAVPALAPAPFTLPLQVAGATNHRASAVPGLGTPELPRADGLALALEPVRATANFLAMVDNLQGDSGRGYYLEMLIGTPPQKVQILVDTGSSNFAVAGAPHSYIDTYFDSESSSTYHSKGFDVTVKYTQGSWTGFVGEDLVTIPKGFNSSFLVNIATIFESENFFLPGIKWNGILGLAYAALAKPSSSLETFFDSLVAQAKIPDIFSMQMCGAGLPVAGSGTNGGSLVLGGIEPSLYKGDIWYTPIKEEWYYQIEILKLEIGGQNLNLDCREYNADKAIVDSGTTLLRLPQKVFDAVVEAVARTSLIPEFSDGFWTGAQLACWTNSETPWAYFPKISIYLRDENASRSFRITILPQLYIQPMMGAGFNYECYRFGISSSTNALVIGATVMEGFYVVFDRAQRRVGFAVSPCAEIEGTTVSEISGPFSTEDIASNCVPAQALNEPILWIVSYALMSVCGAILLVLILLLLLPLHCRHAPRDPEVVNDESSLVRHRWK